(19) United States
(12) Patent Application Publication
(10) Pub. No.: US 2026/0002214 A1
(43) Pub. Date: Jan. 1, 2026

(54) PERSONALIZED TUMOR MARKERS

(71) Applicants: Stichting Het Nederlands Kanker Instituut-Antoni van Leeuwenhoek Ziekenhuis, Amsterdam (NL); Stichting VU, Amsterdam (NL)

(72) Inventors: Gerrit Albert MEIJER, Amsterdam (NL); Evert VAN DEN BROEK, Amsterdam (NL); Remondus Johannes Adriaan FIJNEMAN, Amsterdam (NL); Sanne ABELN, Amsterdam (NL); Soufyan LAKBIR, Amsterdam (NL); Jakob HERINGA, Amsterdam (NL)

(21) Appl. No.: 18/254,732

(22) PCT Filed: Nov. 26, 2021
(86) PCT No.: PCT/NL2021/050720

§ 371 Date: May 26, 2023

(30) Foreign Application Priority Data
Nov. 26, 2020 (EP) 20210101.0

(51) Int. Cl. C12Q 1/6886; A61K 45/06

(52) U.S. Cl. CPC C12Q 1/6886; A61K 45/06; C12Q 2600/156

(57) ABSTRACT

The invention relates to methods for tumor marker analysis comprising providing a genomic DNA sample from tumor cells of a patient, preselecting a chromosomal region on the genomic DNA comprising at least part of a potential structural variant (SV); and sequencing the genomic region to characterize the potential SV. The invention further relates to methods for detecting minimal residual disease, monitoring treatment response and tumor progression in a patient by employing the SV, and to personalized therapy based on the identity of the SV.

### CROSS-REFERENCE TO RELATED PATENT APPLICATIONS

This application is a U.S. National Stage Application under 35 U.S.C. § 371 of International Patent Application No. PCT/NL2021/050720 filed Nov. 26, 2021, which claims the benefit of priority of Patent Application No. EP 20210101.0 filed Nov. 26, 2020, both of which are incorporated by reference in their entireties. The International Application was published on Jun. 2, 2022, as International Publication No. WO 2022/114957A1.

### FIELD

The invention relates to the field of oncology. More specifically, the invention relates to methods for analyzing a tumor marker. The invention provides methods and means for providing novel, personalized tumor markers that may improve disease management of patients.

### 1 INTRODUCTION

Irreversible somatic DNA aberrations that drive tumorigenesis include small nucleotide variants (SNVs), chromosomal somatic numerical copy number aberrations (SCNAs) and structural variants (SVs) (Cancer Genome Atlas Network, 2012. Nature 487:330-337; Beroukhim et al., 2010. Nature 463:899-905; Li et al., 2020. Nature 578:112-121; Priestley et al., 2019. Nature 575:210-216). SVs include deletions, insertions, inversions, and intra- and inter-chromosomal translocations (Stratton et al., 2009. Nature 458:719-724) and are characterized by chromosomal breaks (Edwards, 2010. J Pathol 220:244-254; Alkan et al., 2011. Nat Rev Genet 12:363-376). Somatic SVs are highly tumor-specific aberrations. While several computational methods are available to detect numerical SCNAs of chromosomal segments in tumor genomes, computational methods were lacking that systematically detect structural chromosomal aberrations by virtue of the genomic location of SCNA-associated chromosomal breaks and identify genes that appear non-randomly affected by chromosomal breakpoints across (large) series of tumor samples.

Colorectal cancer (CRC) develops via a benign precursor lesion, an adenoma. Only a small proportion of approximately 5% of colorectal adenomas is estimated to progress into a malignant lesion (Muto et al, 1975. Cancer 36:2251-2270). The adenoma-to-carcinoma sequence is accompanied by the accumulation of somatic DNA alterations that enables the tumor to evolve (Fearon and Vogelstein, 1990. Cell 61:759-767) and to gain malignant potential by acquiring biological properties that are often referred to as ‘the hallmarks of cancer’, such as self-sufficiency in growth signals, lack of apoptosis, tissue invasion and metastasis, evading the immune system, sustained angiogenesis and limitless replicative potential (Hanahan and Weinberg, 2011. Cell 144:646-674). We extensively investigated the role of SNVs and SCNAs in colorectal adenoma-to-carcinoma progression (Sillars-Hardebol et al., 2012. J Pathol 226:1-6; Carvalho et al., 2009. Gut 58:79-89; Sillars-Hardebol et al., 2012. Gut 61:1568-1575; de Groen et al., 2014. Genes Chromosom Cancer 53:339-348). In contrast, the impact of somatic SVs on tumor progression and clinical behavior is relatively unknown.

There is thus a need to study a role of somatic SVs on tumor progression and clinical behavior.

### 2 BRIEF DESCRIPTION OF THE INVENTION

Cancer is caused by somatic DNA alterations, i.e. SNVs, SCNAs and SVs, which can be used in clinical practice as diagnostic, prognostic, predictive, and disease monitoring biomarkers. Detection of SNVs in (panels of commonly mutated) genes of interest and detection of SCNAs is established in routine molecular diagnostics clinical practice.

### SVs are Highly Prevalent

An algorithm, termed ‘GeneBreak’ has been developed (van den Broek et al., 2016. F1000Res 5:2340) to systematically identify genes recurrently affected by the genomic location of chromosomal SCNA-associated breaks by a genome-wide approach, which can be applied to DNA copy number data obtained by array-Comparative Genomic Hybridization (aCGH) or by (low-pass) whole genome sequencing (WGS). GeneBreak was now applied to a series of 352 aCGH profiles from primary CRCs that ultimately metastasized for which we demonstrated the high prevalence of 748 gene regions with recurrent SVs, among which MACROD2 that was affected by SVs in 41% of the samples (van den Broek et al., 2015. PLOS One 10: e0138141). Likewise, also in a series of 114 stage II and III non-metastatic microsatellite stable (MSS) colon cancers SV recurrences were highly prevalent (van den Broek et al., 2016: Oncotarget 7:73876-73887). An aspect of the present invention is the concept that these recurrent SVs can be used as personalized tumor markers.

The invention therefore provides a method for tumor marker analysis comprising providing a genomic DNA sample from tumor cells of a patient; preselecting a chromosomal region on the genomic DNA comprising at least part of a potential structural variant (SV); and sequencing the genomic region surrounding at least one chromosomal breakpoint of the potential SV, to provide one or more novel tumor markers that can be used to identify tumor cells of the patient and/or that are specific for said tumor of the patient. Said genomic DNA sample may be obtained from fresh tissue, such as fresh frozen tissue, or obtained from fixed tissue, such as formalin-fixed paraffin-embedded (FFPE) tissue.

The step of preselecting in methods of the invention may be performed by capturing and isolating the chromosomal region comprising at least part of a potential structural variant (SV), for example by Targeted Locus Amplification (TLA).

Following the step of preselecting, the nucleotide sequence of the genomic region surrounding the potential SV may be determined, preferably by third generation sequencing.

In methods of the invention, the potential SV is a recombination hotspot, for example within one or more genes listed in Table 2. Said potential SV may comprise a recombination hotspot within one or more of the genes MACROD2, FHIT, RBFOX1, PARK2, TTC28, NOTCH2, PIBF1, CCSER1, PTPRN2, NAALADL2, WWOX, and PRKG1.

In methods of the invention, the potential SV is caused by activation of a retrotransposon, preferably a LINE1 element, preferably a hot-L1 element, for example within one or more genomic regions listed in Table 3.

Said potential SV may comprise a region on chromosome 22, from nucleotide 29062500 to nucleotide 29070000; a region on chromosome 23, from nucleotide 11730000 to nucleotide 11737500; a region on chromosome 14, from nucleotide 59220000 to nucleotide 59227500; a region on chromosome 12, from nucleotide 3607500 to nucleotide 3615000; a region on chromosome 7, from nucleotide 57442500 to nucleotide 57450000; a region on chromosome 8, from nucleotide 143955000 to nucleotide 143962500; a region on chromosome 9, from nucleotide 139995000 to nucleotide 140002500; a region on chromosome 12, from nucleotide 132060000 to nucleotide 132067500; a region on chromosome 6, from nucleotide 170482500 to nucleotide 170490000; a region on chromosome 5, from nucleotide 742500 to nucleotide 750000, or a combination thereof.

The invention further provides a method of typing a sample from a cancer patient, the method comprising providing a sample comprising nucleic acids from said cancer cells; determining a number of structural variants (SV) in said sample; comparing said number of SV to a number of SV in a reference; and typing said sample based on the comparison of the number of SV. Said method may further comprise determining presence or absence of an SV that affects exon sequences of MACROD2, and/or presence or absence of mutations in TP53.

The invention further provides a method for monitoring tumor progression in a patient, comprising identifying one or more novel tumor markers that are specific for said tumor of the patient as a structural variant (SV) in the patient by performing a method of the invention, whereby the SV is characterized by a region of at least 20 nucleotides at either site of the SV's associated chromosomal breakpoint, providing a first biopsy from the patient, analyzing the first biopsy for absence or presence and/or abundance of the SV, providing a second biopsy from the patient, whereby the provision of the second biopsy is separated in time or location from the provision of the first biopsy, analyzing the second biopsy for presence or abundance of the SV, and recording and comparing presence or abundance of the SV in the two biopsies.

Said biopsy preferably is a liquid biopsy, more preferably a blood sample. In methods for monitoring tumor progression, the patient is preferably treated by therapy between the first and second biopsy. Said therapy may be selected from surgery, chemotherapy, radiation therapy, targeted therapy, immunotherapy, stem cell or bone marrow transplant, hormone therapy, or a combination thereof.

The invention further provides immunotherapy, including an immune checkpoint inhibitor such a PD1/PDL1 inhibitor and/or a CTLA-4 inhibitor, for use in a method of treating of a cancer patient with a structural variant (SV) in at least one of WWOX, FHIT, GMDS and PIBF1.

The invention further provides capecitabine and oxaliplatin, optionally combined with a vascular endothelial growth factor inhibitor such as bevacizumab, ziv-aflibercept, or ramucirumab, an epidermal growth factor receptor inhibitor such as cetuximab or panitumumab, irinotecan, trifluridine and tipiracil, or a combination thereof, for use in a method of treating a cancer patient with a SV in MACROD2, especially an SV that affects exon sequences of MACROD2.

### 3 FIGURE LEGENDS

FIG. 1. Schematic overview of SCNA-associated chromosomal break calling. SCNA copy number data are called on TCGA data (The Cancer Genome Atlas Network, 2012. Nature 487:330-337), segments of chromosomal gains or losses are indicated by horizontal lines in the ‘copy number calling’ plot. Chromosomal breaks are positioned where DNA copy number segments change, i.e. at the border between two adjacent segments. The calling of SCNA-associated breaks was applied to DNA from normal (white blood) cells from cancer patients and on tumor tissue, allowing to distinct ‘noise’ from ‘signal’. Based on the ‘normal’ versus ‘tumor’ comparison, two metrics were used to filter background noise in the data: 1) the smallest adjacent segment size (SAS), and 2) the break size (BS). The smallest adjacent segment size is the probe count of the smallest segment adjacent to the chromosomal break. The SAS filtering was applied to exclude chromosomal breaks called due to small segments with highly altered copy number variation (upper left area). The break size is the absolute difference in DNA copy number between the segments adjacent to the chromosomal break. The BS filtering was applied to exclude chromosomal breaks called due to large segments with very limited copy number variation (lower right area). Chromosomal breaks with a SAS<20 or a BS<0.135 were excluded. In this way, chromosomal breaks present in normal tissue samples were filtered out from the list of unbalanced somatic chromosomal breaks. The top-right area of the tumor plot is indicative for SCNA-associated chromosomal breaks and yielded the data that were used for further analyses.

FIG. 2. (A) Schematic overview of the construction of a Tumor Break Load (TBL) classifier using gene expression data. TBL is defined as the sum of SCNA-associated SVs, i.e. unbalanced somatic chromosomal breaks per tumor sample. The upper and lower quantiles of the TBL distribution are used as ‘High TBL’ and ‘Low TBL’ groups. (B) The model is trained using gene expression data to predict the TBL phenotypic status (‘High TBL’ or ‘Low TBL’). The trained model is used to classify all TCGA COADREAD microsatellite stable (MSS) patients in a ‘Low TBL’ or ‘High TBL’ expression phenotype. (C) The classified TBL status is used for survival analysis.

FIG. 3. Left panels-Density curves overlaying histograms of the natural log transformed tumor break load (In (TBL)) for TCGA stage I-IV COADREAD MSS colon and rectal cancer samples (A), stage I-IV BRCA breast cancer samples (B) and stage I-IV LUAD lung cancer samples (C). Right panels-Volcano plots indicating the TBL grouped per pathological stage. The Median TBL is annotated at the right side of the volcano plot. Statistical significance is determined using the Wilcoxon signed-rank test. P-values<0.05 were considered significant. Group sizes denoted with N.

FIG. 4. Kaplan-Meier curves of TCGA COADREAD MSS patients for the classified TBL groups, including Stage I-IV (A) or focusing on localized Stage I-III disease (B). ‘TBL-high’ expression phenotype patients tend to have a worse prognosis (lower line) compared to ‘TBL-low’ expression phenotype patients (upper line), as indicated by the considerably large Hazard Rates (HR). The number of patients (N) are shown in parentheses. Hazard rates were estimated using a Cox proportional-Hazards model. Statistical significance was measured using a Log-rank test. P-values<0.05 were considered significant.

FIG. 5. Gene-centric chromosomal break (SCNA-associated SVs) frequency ranking for TCGA MSS COADREAD (A), BRCA (B), and LUAD (C) samples. The top 10 genes are annotated at their respective position.

FIG. 6. Comparison of the TBL in COADREAD MSS samples in which MACROD2 was not affected by SCNA-associated SVs (‘WT’) to the samples in which MACROD2 was affected by SCNA-associated SVs (‘Broken’). A highly significant difference in TBL was observed, implying that SVs in MACROD2 are strongly associated with an increased TBL. Statistical significance was estimated using a Wilcoxon signed-rank test. P-values<0.05 were considered significant. Group sizes denoted with N.

FIG. 7. The AUC of models trained to predict the unbalanced chromosomal break status (SCNA-associated SVs) of a given gene from the gene expression profile of the corresponding sample. The genes that are shown here comprise the genes most frequently affected by SVs in TCGA data (FIG. 5A) combined with the genes most frequently affected by SVs in CRCs compared to adenoma precursor lesions (van den Broek, thesis chapter 5, VU University Medical Center 16 Dec. 2016). For the well-known (colorectal) cancer genes APC, KRAS, and TP53, the AUC of models predicting SNV mutation status were taken along for comparison, as a reference. AUC of 0.5 denoted with a dashed line as indication of random performance.

FIG. 8. Overview of the CRC SV distribution as called by the Hartwig Medical Foundation (HMF). (A) Every vertical bar denotes a single biopsy. Samples are ordered along the x-axis ranging from high to low total number of SVs. The y-axis shows the number of SVs. In panel (a) all SV types are shown together. The boxplot shows the median and the interquartile range (IQR). The whiskers have a maximum of 1.5 IQR, with outliers shown as dots above. (B) The panels show the SVs per type with the sample-ordering along the x-axis maintained as shown in panel (A). Note the high amount of translocation and deletion calls.

FIG. 9. Example of targeted detection of an SV in FHIT on chromosome 3, visualized using the Ribbon interface. (A) Overview of the long reads spanning the genomic position where the SV call is made. The reference genome is shown at the top. Two parts of chromosome 3 are expanded below. The black box shows the genomic position of the SV call. Three out of the fifteen reads support an SV by aligning up to the black box and then ‘jumping’ 35 Mb where they align with a different part of chromosome 3. (B) Expanded overview of the alignment of the read marked in bold. This particular translocation implies that the FHIT gene is truncated to only its first 5 exons, which likely affects its function.

FIG. 10. Kaplan Meier curves of the disease-free survival (DFS) of stage II-III TCGA COADREAD MSS patients for TBL-High and TBL-Low groups, defined by a TBL cut-off of 54 (see Methods). TBL-high patients show a significantly worse prognosis (lower black line) compared to TBL-low patients (upper grey line), as indicated by the large Hazard Rate (HR). The number of patients (N) are shown in parentheses. Hazard rates were estimated using a Cox proportional-Hazards model. Statistical significance was determined using a Log-rank test. P-values<0.05 were considered significant.

FIG. 11. Kaplan Meier curves of the Relapse Free Survival (RFS) of stage II-III MSS colorectal cancer patients (dataset Orsetti,B. et al., Impact of chromosomal instability on colorectal cancer progression and outcome. BMC Cancer, 14, 121 (2014)) for TBL-High and TBL-Low groups, defined by a TBL cut-off of 78 (see Methods). TBL-high patients show a significantly worse prognosis (lower black line) compared to TBL-low patients (upper grey line), as indicated by the large Hazard Rate (HR) validating our observations from the TCGA data (see Figure A). The number of patients (N) are shown in parentheses. Hazard rates were estimated using a Cox proportional-Hazards model. Statistical significance was measured using a Log-rank test. P-values<0.05 were considered significant.

FIG. 12. Violin boxplots of the association between the genomic position of SVs in MACROD2 with the Tumor Break Load (TBL). SVs affecting exon 5 or 6 of MACROD2 are associated with a significantly higher TBL compared to CRCs in which the SVs only affect MACROD2 intronic sequences or CRCs without MACROD2 alterations.

FIG. 13. Violin boxplots indicating the association between somatic alterations in TP53 and/or MACROD2 with the Tumor Break Load (TBL) in stage I-IV MSS CRC patients (TCGA COADREAD). CRCs with alterations in MACROD2 (SVs) or TP53 (SNVs) have a significantly higher TBL than CRCs without alterations in either of these genes. The effect on TBL is highest when both MACROD2 and TP53 are mutated. The median TBL is reported next to the boxplots. Differences in TBL between the various TP53 and MACROD2 mutation states have been assessed with a Wilcoxon-signed rank test. P<0.05 is considered significant. Group size denoted with N.

FIG. 14. Ranking of genes by the frequencies in which they are affected by (SCNA-associated) SVs. (A) TCGA COADREAD MSS tumors (excluding MSI as well as POLD/E mutants); (B) TCGA COADREAD MSI tumors. The prevalence of genes affected by SVs is dependent on the genomic instability phenotype (MSS or MSI) of the tumour. For instance, WWOX is significantly more frequently affected in MSI COADREAD tumors compared to MSS COADREAD tumors (p<0.0001). This implies that alterations in these genes are not just occurring as bystander effects of tumor development, but in fact have a biological function. The top 10 most frequently affected genes are annotated at their respective position. Genes that are differentially affected by SVs in MSS versus MSI samples are listed in Table 7.

### 4 DETAILED DESCRIPTION OF THE INVENTION

### 4.1 Definitions

The term “lesion”, as is used herein, refers to a cancerous growth of epithelial tissue that covers or lines surfaces of the colorectal tract. Said cancerous growth preferably is an adenocarcinoma. The term lesion includes reference to adenoma, early adenoma, advanced adenoma, low-risk adenoma, high-risk adenoma and colorectal cancer.

The term “adenoma”, as is used herein, refers to a benign tumor of epithelial tissue with glandular origin, glandular characteristics, or both. Said adenoma preferably is a colorectal adenoma, also referred to as an adenomatous polyp.

The term “protein expression molecule”, as is used herein, refers to a protein product of a gene, or a part of such product.

A “detectable label” is a label which may be detected and of which the absolute or relative amount and/or location (for example, the location on an array) can be determined.

The term “specifically binding”, as is used herein, refers to a binding reaction between an antibody-antigen, or other binding pair, which is determinative of the presence of a protein comprising the antigen in a heterogeneous population of proteins and/or other biologics. Thus, under designated conditions, a specified antibody or functional part thereof binds to a particular antigen and does not bind in a significant amount to other proteins present in the sample.

The term “polypectomy”, as is used herein, refers to the partial or complete removal of an adenoma.

The term “enzyme-linked immunosorbent assay (ELISA)”, as is used herein, refers to a plate-based assay that is designed for detecting and quantifying antigens such as protein expression molecules.

The term “preselecting”, as is used herein in the context of preselecting a chromosomal region, refers to the sufficient isolation of the chromosomal region such that it can be sequenced. The term “preselecting” includes “pulling down”, “capturing”, “enriching for”, “isolating” and/or “circularizing” of the chromosomal region to such extent that a nucleotide sequence of the chromosomal region can be determined.

The term “fixed tissue”, as is used herein, refers to the preservation of biological tissues. Tissue fixation is a critical step in the preparation of histological sections. Fixation may be performed using any technology selected from heat fixation, chemical fixation using, for example, a crosslinking fixative such as an aldehyde including formaldehyde, paraformaldehyde, glutaraldehyde, and combinations thereof, alcohol such as ethanol and methanol, acetone, acetic acid, potassium dichromate, chromic acid, and potassium permanganate, mercurials such as Zenker's fixative, picrates such as Bouin's solution, and Hepes-glutamic acid buffer-mediated organic solvent protection effect (HOPE; Polysciences, Inc., Warrington, PA). A preferred fixative is formalin, an aqueous solution of formaldehyde.

The term “structural variant (SV)”, as is used herein, refers to a chromosomal aberration, including one or more insertions, duplications, deletions, inversions and/or translocations, involving at least one double stranded chromosomal break (Escaramís et al., 2015. Briefings Funct Genom 14:305-314; Feuk et al., 2006. Nature Rev Gen 7:85-97). Insertions are the introduction of a novel sequence with respect to a reference genome. Duplications are the introduction of an existing sequence with respect to a reference genome. Deletions are the loss of sequence with respect to a reference genome. Inversions are segments of DNA that are in reverse order with respect to a reference genome. Chromosomal translocations are defined as a change in position of a DNA sequence, and can be either intra- or inter-chromosomatic. The term SV refers to the presence of a chromosomal aberration. Said SV may be characterized by a region of at least 10 nucleotides at either site of at least one of the SV's associated chromosomal breakpoint or associated chromosomal breakpoints.

The term “tumor marker”, as is used herein, refers to a region of at least 10 nucleotides at either site of an SV-associated chromosomal breakpoint. The term tumor marker preferably refers to a nucleotide sequence comprising the breakpoint of an SV and including at least 10 nucleotides at either side of the breakpoint, preferably between 10 and 2000 nucleotides, such as between 20 and 1000 nucleotides, including 50, 100, 200, and 500 nucleotides on each side of the breakpoint. A personalized tumor marker is specific for a patient, meaning that the position of the breakpoint and/or the nucleotide sequences surrounding the breakpoint, are unique for a specific tumor in a specific patient. A personalized tumor marker allows personalized monitoring of a patient, for example during or after treatment.

The term “bin”, as is used herein, refers to a genomic subregion. For many calculations, the genome can be divided into small regions (bins), on which the calculations are actually performed. A bin may be a fixed genomic region of, for example, 5 kilobases (kb), 10 kb, or 15 kb.

The term “small nucleotide variant (SNV)”, as is used herein, refers to an alteration of one or a few nucleotides, such as two or three nucleotides, at a specific position in the genome. Said SNV is not present in a reference genome sequence. The term “small nucleotide variant” is preferred over the term “single nucleotide variant” to cover the occurrence of several single nucleotide alterations within a short genomic region of less than 10 base pairs, preferably less than 5 base pairs.

The term “Targeted Locus Amplification (TLA)”, as is used herein, refers to crosslinking and circularization of DNA fragments that were in physical proximity to each other and, therefore, were in the same chromosomal region on the genomic DNA.

The term “third generation sequencing”, as is used herein, refers to long-read sequencing techniques such as provided by Oxford Nanopore Technologies and Pacific Biosciences.

The term “retrotransposon”, as is used herein, refers to a mobile element that may form an intermediate RNA transcript from which a DNA copy of is made using a reverse transcriptase and inserted into the genome at a new location. There are about 31 human endogenous retrotransposon families extant in the human genome, including Long Interspersed Nuclear Elements (LINEs) and Short Interspersed Nuclear Elements (SINEs). LINE-1 is currently the only known active autonomous mobile element in human.

The term “hot-L1”, as is used herein, refers to a small subset of LINE11 elements that are usually transcriptionally repressed, but which may be activated and subsequently retrotranspose due to epigenetic changes that may occur in tumors (Rodriguez-Martin et al., 2020. Nature Gen 52:306-319).

The term “recombination hotspot”, as is used herein, refers to a region on a genome that exhibits elevated rates of recombination relative to a neutral expectation. The recombination rate within hotspots can be hundreds of times that of the surrounding region (Jeffreys et al., 2001Nat Genet 29:217-22).

The term “tumor break load (TBL)”, as is used herein, refers to the sum of SVs per tumor sample, whereby an SV is characterized by the presence of at least one double stranded chromosomal break as is indicated herein above.

The terms “high TBL” and “low TBL”, as are used herein, refer to samples in the upper and lower quantiles of a TBL distribution, respectively, preferably the upper and lower quartiles of a TBL distribution. Said high or low TBL may be determined using gene expression data, for example with the use of a trained classifier model, to predict a TBL phenotypic status as ‘high TBL expression phenotype’ or ‘low TBL expression phenotype’.

The term “nucleic acid mimetic”, as is used herein, refers to a chemically-modified DNA and RNA molecule which exhibits enhanced stability, bioavailability, specificity and/or efficacy. Said chemically-modified, or synthetic, DNA and RNA molecule comprises the use of modified building blocks such as a 3′-modified, N3′->P5′ phosphoramidate analogue, the use of modifications at the 2′-position of nucleoside sugar rings, the use of bridged nucleotides, such as 2′4′-bridged or 2′,4′-BNANC (2′-O,4′-aminoethylene bridged) nucleotides, locked nucleic acid molecules (LNA) peptide nucleic acid molecules (PNA), or combinations thereof.

The term “reference”, as is used herein, refers to a numerical value that functions as a cut-off value to differentiate between Tumor Break Load (TBL)-high patients with poor prognosis and TBL-low patients with good prognosis, using methods that are known to a person skilled in the art, such as statistical-mathematical methods that derive the optimal cut-off, e.g. OptimalCutpoints, maxstat or the Youden Index derived from a Receiver Operator Characteristic (ROC) curve, summary statistics such as the quantiles or median, or machine learning methods that classify from a panel of targets, using data derived from a group of patients with similar tumor stage and/or similar technology/methodology to determine TBL. As is known to a person skilled in the art, the actual value will differ per tumor and stage of the tumor, and per technology/methodology that was used to determine the TBL. As an example, said reference may be a numerical value between about 1 and about 3000, such as 1, 2, 3, 5, 10, 25, 50, 30, 40, 50, 51, 52, 53, 54, 55, 56, 57, 58, 59, 60, 70, 80, 90, 100, 250, 500, 1000, or 3000. The term further includes a physical reference such as a nucleic acid sample isolated from a normal tissue of a cancer patient, such as a nucleic acid sample from white blood cells, normal adjacent tissue from a cancer patient or tissue of a healthy individual, including a nucleic acid sample from white blood cells or normal tissue that is pooled from two or more patients.

The term “liquid biopsy”, as is used herein, refers to a liquid sample that is obtained from an individual. Said liquid biopsy is preferably selected from blood, urine, milk, cerebrospinal fluid, peritoneal fluid, interstitial fluid, lymph, amniotic fluid, bile, cerumen, feces, female ejaculate, gastric juice, mucus pericardial fluid, pleural fluid, pus, saliva, semen, smegma, sputum, synovial fluid, sweat, tears, vaginal secretion, and vomit. A preferred liquid biopsy is blood.

The term “mono-ADP ribosylhydrolase 2 (MACROD2)”, as is used herein, refers to a gene that encodes a deacetylase involved in removing ADP-ribose from mono-ADP-ribosylated proteins. The encoded protein has been shown to translocate from the nucleus to the cytoplasm upon DNA damage. The gene resides on chromosome 20 and is characterized by HGNC entry code 16126, Entrez Gene entry code 140733 and Ensembl entry code ENSG00000172264. The encoded protein is characterized by UniProt entry code A1Z1Q3.

The term “Fragile Histidine Triad diadenosine triphosphatase (FHIT)”, as is used herein, refers to a gene that encodes a diadenosine P1,P3-bis(5′-adenosyl)-triphosphate adenylohydrolase that is involved in purine metabolism. The gene resides on chromosome 3 and is characterized by HGNC entry code 3701, Entrez Gene entry code 2272 and Ensembl entry code ENSG00000189283. The encoded protein is characterized by UniProt entry code P49789.

The term “RNA Binding Fox-1 Homolog 1 (RBFOX1)”, as is used herein, refers to a gene that encodes a protein that is involved in tissue-specific alternative splicing. The gene resides on chromosome 16 and is characterized by HGNC entry code 18222, Entrez Gene entry code 54715 and Ensembl entry code ENSG00000078328. The encoded protein is characterized by UniProt entry code Q9NWB1.

The term “Parkinson Disease (Autosomal Recessive, Juvenile) 2 (PARK2)”, as is used herein, refers to a gene that encodes a component of a multiprotein E3 ubiquitin ligase complex that mediates the targeting of substrate proteins for proteasomal degradation. The gene resides on chromosome 6 and is characterized by HGNC entry code 8607, Entrez Gene entry code 5071 and Ensembl entry code ENSG00000185345. The encoded protein is characterized by UniProt entry code 060260.

The term “Tetratricopeptide Repeat Domain 28 (TTC28)”, as is used herein, refers to a gene that encodes a protein that may be involved in condensation of spindle midzone microtubules, leading to the formation of midbody. The gene resides on chromosome 22 and is characterized by HGNC entry code 29179, Entrez Gene entry code 23331 and Ensembl entry code ENSG00000100154. The encoded protein is characterized by UniProt entry code Q96AY4.

The term “Coiled-Coil Serine Rich Protein 1 (CCSER1)”, as is used herein, refers to a gene that encodes a protein with unknown function. The gene resides on chromosome 4 and is characterized by HGNC entry code 29349, Entrez Gene entry code 401145 and Ensembl entry code ENSG00000184305. The encoded protein is characterized by UniProt entry code Q9C013.

The term “Protein Tyrosine Phosphatase Receptor Type N2 (PTPRN2)”, as is used herein, refers to a gene that encodes a potential phosphatidylinositol phosphatase with the ability to dephosphorylate phosphatidylinositol 3-phosphate and phosphatidylinositol 4,5-diphosphate. The gene resides on chromosome 7 and is characterized by HGNC entry code 9677, Entrez Gene entry code 5799 and Ensembl entry code ENSG00000155093. The encoded protein is characterized by UniProt entry code Q92932.

The term “N-Acetylated Alpha-Linked Acidic Dipeptidase Like 2 (NAALADL2)”, as is used herein, refers to a gene that encodes a protein with unknown function. The gene resides on chromosome 3 and is characterized by HGNC entry code 23219, Entrez Gene entry code254827 and Ensembl entry code ENSG00000177694. The encoded protein is characterized by UniProt entry code Q58DX5.

The term “WW Domain Containing Oxidoreductase (WWOX)”, as is used herein, refers to a gene that encodes a protein that may act as a tumor suppressor and may play a role in apoptosis. The gene resides on chromosome 16 and is characterized by HGNC entry code 12799, Entrez Gene entry code 51741 and Ensembl entry code ENSG00000186153. The encoded protein is characterized by UniProt entry code Q9NZC7.

The term “Protein Kinase CGMP-Dependent 1 (PRKG1)”, as is used herein, refers to a gene that encodes a cyclic GMP-dependent protein kinase which acts in the nitric oxide/cGMP signaling pathway. The gene resides on chromosome 10 and is characterized by HGNC entry code 9414, Entrez Gene entry code 5592 and Ensembl entry code ENSG00000185532. The encoded protein is characterized by UniProt entry code Q13976.

The term “NOTCH2”, as is used herein, refers to a gene that encodes a type 1 transmembrane protein that functions as a receptor for membrane-bound ligands Jagged-1, Jagged-2 and Delta-1 to regulate cell-fate determination. The gene resides on chromosome 1p12 and is characterized by HGNC entry code 7882, Entrez Gene entry code 4853 and Ensembl entry code ENSG00000134250. The encoded protein is characterized by UniProt entry code Q04721.

The term “Progesterone Immunomodulatory Binding Factor 1 (PIBF1)”, as is used herein, refers to a gene that is induced by the steroid hormone progesterone and plays a role in the maintenance of pregnancy. The gene resides on chromosome 13q21 and is characterized by HGNC entry code 23352, Entrez Gene entry code 10464 and Ensembl entry code ENSG00000083535. The encoded protein is characterized by UniProt entry code Q8WXW3. The term “Poly(ADP-Ribose) Polymerase Family Member 8 (PARP8)”, as is used herein, encodes a mono-ADP-ribosyltransferase that mediates mono-ADP-ribosylation of target proteins. The gene resides on chromosome 5q11 and is characterized by HGNC entry code 26124, Entrez Gene entry code 79668 and Ensembl entry code ENSG00000151883. The encoded protein is characterized by UniProt entry code Q8N3A8.

### 4.2 Analysis of Tumor Markers

Oncogene activation and tumor suppressor gene inactivation can be caused by several classes of somatic DNA aberrations, including small nucleotide variants (SNVs), chromosome-segment somatic copy number aberrations (SCNAs) and chromosomal breakpoint structural variants (SVs) (Stratton et al., 2009. Nature 458:719-724). SVs represent deletions, insertions, inversions, and intra- and inter-chromosomal translocations, all of which involve chromosomal breaks (Edwards, 2010. J Pathol 220:244-254). Interestingly, while SNVs and SCNAs have been examined extensively, genes that are affected by chromosomal breakpoint SVs are poorly characterized, on the one hand because SCNA-associated SVs were considered random events driven by SCNAs and on the other hand due to lack of availability of deep whole genome sequencing (WGS) data and bioinformatic pipelines suited for SV detection. Consequently, the impact of SVs on tumor development and patient clinical outcome is currently highly underestimated.

In one aspect, the invention is directed to a method for tumor marker analysis, especially personalized tumor marker analysis. The inventors have identified potential genomic structural variants that are often prone to alterations, especially in colorectal lesions. The identification of one or more of these genomic structural variants in tumor cells of a patient will allow personalized monitoring of said patient, for example during or after treatment.

The invention therefore provides a method for tumor marker analysis, comprising providing a genomic DNA sample from tumor cells of a patient, preselecting a chromosomal region on the genomic DNA comprising at least part of a potential structural variant (SV), and sequencing the genomic region to determine presence of the SV and to characterize the SV, when present. It is noted that the act of removing a tumor, or part of a tumor, from a patient is not part of this invention.

Said patient may suffer, or is expected to suffer, from a lesion. The analysis of one or more specific tumor markers in said lesion will allow monitoring progression of said lesion, for example from adenoma to colorectal cancer, and/or monitoring a response to treatment. In addition, said one or more specific tumor markers will allow monitoring tumor cells from said patient using a liquid biopsy, thereby providing routine, minimally-invasive and highly-sensitive monitoring for response to cancer treatment and recurrence surveillance. In addition, said one or more specific tumor markers will allow to guide (neo) adjuvant treatment selection by predicting drug sensitivity.

Most molecular tests are performed on fresh and/or fresh frozen tissues such as a blood sample or a biopsy. In most clinical molecular pathology settings, fresh frozen issues are rare, due to the complexities of the logistic chain for the preparation, collection and storage of such samples. Instead, FFPE is the method of choice and often the gold standard for clinicians. FFPE specimen are much easier to prepare and to store, but it is well established that formalin fixation results in DNA damage. Formaldehyde reacts with DNA and proteins, resulting in cross-linked DNA-DNA, DNA-RNA, and DNA-protein complexes. Formaldehyde is also known to induce oxidation and deamination reactions and the formation of cyclic bases derivatives. These chemical modifications have the potential to alter molecular testing through inhibition of enzymatic reparation of DNA or direct changes at single base or sequence levels. Furthermore, crosslinks lead to DNA fragmentation that render sequencing and analysis even more complicated.

The identification of personalized tumor markers is especially challenging for fixed tissue, such as formalin-fixed tissue. The fragmented and cross-linked genomic DNA in fixed tissue barely allows individualization and identification by sequencing of a chromosomal region surrounding a potential structural variant (SV), which could provide a novel, personalized tumor marker.

The methods of the invention provide the preselection of a chromosomal region comprising at least part of a potential structural variant (SV). In the present context, preselecting a chromosomal region refers to the sufficient isolation of a chromosomal region such that it can be sequenced.

Preselection may be performed by capturing a potential SV, for example by employing probes. For this, several methods are commercially available, including SureSelect® Custom Target Enrichment Library Preparation (Agilent, Santa Clara, CA), HaloPlex™ Target Enrichment System (Agilent, Santa Clara, CA), Illumina DNA Prep with Enrichment (Nextera Flex for Enrichment; Illumina, San Diego, CA), and SeqCap EZ Choice Library Preparation (Roche/Nimblegen, Madison, WI).

In general, genomic DNA is fragmented, for example by sonication, restriction enzyme cutting and/or transposase activity, followed by capturing of genomic sequences by probe hybridization and amplification of the captured genomic DNA following adapter ligation. The average insert size of a resulting library preferably is 150-700 nucleotides, and routinely is 200-400 nucleotides.

Probes that are specific for a potential SV-associated chromosomal breakpoint preferably are 20-150 nucleotides, such as 25-105 nucleotides, such as 25 nucleotides, 30 nucleotides, 50 nucleotides, 75 nucleotides, or 100 nucleotides. Said probe preferably is a single stranded nucleic acid molecule, such as a DNA or RNA molecule, or a single stranded nucleic acid mimetic.

To cover a substantially complete potential SV-associated chromosomal breakpoint, several probes may be used that target the potential SV-associated chromosomal breakpoint. Said probes preferably may be gapped or tiled, and may comprise overlapping nucleotides.

Preselection may also be performed by crosslinking of the genomic DNA prior to fragmenting said genomic DNA. Crosslinking mainly will occur between genomic sequences that are in the vicinity of each other. The procedure of crosslinking and fragmenting DNA within a cell, and subsequent ligation of cross-linked DNA fragments, may provide an ideal starting point for preselecting and subsequent sequencing of a genomic region of interest such as an SV.

Targeted Locus Amplification (TLA) is an example of such crosslinking approach. TLA has been described in WO2012/005595, which is herein incorporated by reference in its entirety. TLA comprises building a contig of a genomic region of interest comprising a target nucleotide sequence, the method comprising fragmenting a crosslinked DNA, ligating the fragmented cross linked DNA, reversing the crosslinking and determining at least part of the sequences of ligated DNA fragments, comprising a DNA fragment with the target nucleotide sequence, and using the determined sequences to build a contig of the genomic region of interest.

Following preselection, a genomic region encompassing the potential SV is sequenced. Sequencing may be performed using any method available in the art, including classical DNA sequencing technologies, such as Sanger sequencing and Maxam and Gilbert sequencing, as well as next generation sequencing (also referred to as massively parallel sequencing) technologies, such as Ion Torrent sequencing and Illumina sequencing and, preferably, third generation sequencing technologies such as Nanopore sequencing and PacBio® sequencing.

Algorithms such as ‘GeneBreak’ (van den Broek et al., 2017. F1000Research 5: 2340) may help to determine the genomic positions of chromosomal breakpoints, reasoning that intra-chromosomal changes in SCNA-status can only be explained by mechanisms that involve chromosomal breaks. Rather than acting as passenger mutations, recent publications may support a driver function for such SVs. For instance, MACROD2 mutations cause chromosomal instability and thereby promote colon tumor progression (Jin and Burkard, 2018. Cancer Discov 8:921-923, Sakthianandeswaren et al., 2018. Oncotarget 9:33056-33058; Sakthianandeswaren et al., 2018. Cancer Discov 8:988-1005).

### 4.3 Potential Structural Variant (SV)

The invention is based on the recognition that certain genomic structural variants, comprising genomic recombination hotspots and mobilized transposable elements, are able to provide unique, personalized tumor markers for the majority of tumors in individual patients. Preselection and sequence analysis of a genomic region surrounding a structural variant may provide one or more novel tumor markers that are specific for a tumor of an individual patient. There may be some overlap between genomic recombination hotspots and mobilized transposable elements, as some of the genomic recombination hotspots are prone to insertion of a mobilized transposable element.

One of these potential SVs comprises a recombination hotspot. Said recombination hotspot may be within one or more genes listed in Table 2. Said recombination hotspot preferably is within one or more genes selected from MACROD2, FHIT, RBFOX1, PARK2, TTC28, NOTCH2, PIBF1, CCSER1, PTPRN2, NAALADL2, WWOX, and PRKG1.

A second class of potential SV comprises smaller regions in or near genes encoding long non-coding RNA (lncRNAs) or genes like DACT1. Example of said second class of potential SVs are listed in Table 3. This class of SV include TTC28-Inc-HSCB-7:1, Inc-ARHGAP6-6:1, Region close to DACT1, PRMT8, Region close to ZNF716, CYP11B1, IncDPP7-1:1, a region close to SFSWAP and MMP17, Inc-FAM120B-3:1//Lnc-DLL1-2-1, ZDHHC11B, or a combination thereof. A comprehensive compendium of human lncRNAs is available at LNCipedia.org. Said compendium provides a public database for lncRNA sequence and annotation (Volders et al., 2019. Nucleic Acids Res 47: D135-D139).

Said potential SV preferably comprises a region on chromosome 22, from nucleotide 29062500 to nucleotide 29070000; a region on chromosome 23, from nucleotide 11730000 to nucleotide 11737500; a region on chromosome 14, from nucleotide 59220000 to nucleotide 59227500; a region on chromosome 12, from nucleotide 3607500 to nucleotide 3615000; a region on chromosome 7, from nucleotide 57442500 to nucleotide 57450000; a region on chromosome 8, from nucleotide 143955000 to nucleotide 143962500; a region on chromosome 9, from nucleotide 139995000 to nucleotide 140002500; a region on chromosome 12, from nucleotide 132060000 to nucleotide 132067500; a region on chromosome 6, from nucleotide 170482500 to nucleotide 170490000; a region on chromosome 5, from nucleotide 742500 to nucleotide 750000. The origin of this class of potential SV may also be a transposable element such as a “hot-L1”.

A third class of potential SV is generated by a transposable element such as a retrotransposon, preferably a LINE-1 element. It was recently found that about half of all cancers have somatic integrations of retrotransposons, especially of long interspersed nuclear elements (LINE-1) (Rodriguez-Martin et al., 2020. Nature Gen 52:306-319). Aberrant LINE-1 integrations can delete large regions of a chromosome, which may result in the removal of a tumor-suppressor gene. In addition, aberrant integration may result in other structural variants such as translocations and large-scale duplications.

As is shown herein, a large proportion of cancers such as CRCs has active hot-L1 elements. The genomic fragments provided in Table 8 can be specifically targeted for detecting tumor-specific SV biomarkers, including personalized markers.

The identification of such SV-hotspots, selected from one or more of a recombination hotspot, a genomic region in or near lncRNAs, or genes like DACT1, and activated transposable elements, as indicated herein above, in tumors such as colorectal cancer will limit the amount of chromosomal regions that is to be captured to identify a tumor marker comprising one or more SVs.

Said SV occur in most tumors, especially solid tumors such as mesothelioma, melanoma, sarcomas and carcinomas. Said carcinomas include adenoid cystic carcinoma, bladder carcinoma, breast cancer, cervical cancer, colorectal cancer, ductal carcinoma, endometrial cancer, esophageal cancer, gastric cancer, kidney cancer, laryngeal cancer, liver cancer, lung cancer, including small cell and non-small cell lung cancer, nasopharyngeal cancer, oral cancer, ovarian cancer, pancreatic cancer, penile cancer, peritoneal cancer, prostate cancer, renal cell carcinoma, thyroid cancer, vaginal cancer. Although the concept of SV, and the use of SV as personalized tumor biomarkers with biological and clinical relevance, is outlined herein especially for colorectal cancer, it can be applied to other cancers as well. For example, data are provided herein that SV are also present in breast cancer and lung cancer. SV, and especially Tumor Break Load (TBL) may provide a prognostic marker that is associated with progression to metastatic disease in all tumors.

### 4.4 Use of Identified SV

From a cancer molecular diagnostics perspective somatic SVs are attractive targets because they are highly tumor-specific and well-suited for e.g. development of sensitive PCR-based assays. As such, commonly occurring SV events in cancer represent opportunities to develop methods for detection of SV biomarkers with high specificity and sensitivity, with the potential to improve early detection (‘who has cancer’), determination of prognosis (‘who to treat’), predict therapy response (‘how to treat’) and to monitor response to treatment (‘when to adapt treatment regimen’).

At present, clinical, pathological and imaging patient information is insufficient to accurately stratify cancer patients into patient subgroups with low or high risk of disease recurrence (determining prognosis), with good or bad response to a given treatment (predicting therapy responsiveness), and to swiftly adapt treatment when a given therapy fails (disease monitoring, detecting therapy resistance). Somatic DNA alterations in cancer can be detected as DNA mutations in resected tumor tissue or tissue biopsies as well as cell-free circulating tumor DNA (ctDNA) in liquid biopsies, which are minimally invasive and can be obtained longitudinally. Therefore, detection of SVs in tumor tissue DNA and/or in liquid biopsy ctDNA has great potential as biomarker to improve disease management of cancer patients.

Detecting molecular alterations that are causally responsible for disease have highest possible specificity, approaching 100%. In terms of biomarker sensitivity, PCR-based methods like digital PCR (dPCR) (Baker, 2012. Nature Methods 9:541-544) have high sensitivity. At present, several dPCR assays are commercially available for detecting hotspot mutations in oncogenes like KRAS and BRAF. Methodologically, compared to relying on detection of single nucleotide variants (SNVs), detection of chromosomal rearrangements is technically more robust. Clinically, expanding circulating tumor DNA assays for genes that are frequently affected by SNVs with ctDNA assays for detecting SVs will increase the percentage of cancer patients that can be monitored by liquid biopsy assays. For the purpose of detecting minimal residual disease (MRD), increasing the number of putative targets with SV ctDNA assays will increase the sensitivity to detect MRD in a given patient (Heitzer et al., 2019. Nat Rev Gen 20:71-88). Moreover, SVs may have predictive value, e.g. as implicated in case of MACROD2 for treatment with (5FU-based) adjuvant chemotherapy (van den Broek et al., 2018. Oncotarget 9:294454-29452). Therefore, development of cancer-specific ctDNA assays for detection of SVs has both technical and clinical advantages.

As is shown in the examples, the number of SVs may provide an independent prognostic marker for tumor patients. Said prognostic marker can be used to score a risk for disease recurrence. Tumor patients with a high TBL, termed “TBL-high”, were shown to have a significantly worse prognosis compared to TBL-low patients (P<0.01, with a Hazard Rate>6). Hence, the number of SVs, as for example determined by TBL, may discriminate between SV-low or TBL-low patients, which may generally be classified as having a good prognosis, and SV-high or TBL-high patients, which may generally be classified as having a poor prognosis. Patients that are classified as having a poor prognosis, have a higher risk of disease recurrence, as is shown herein in the examples.

An optimal cut-off, whereby patients having a number of breaking points above said cut-off had an unfavourable outcome compared to patients that were below this cut-off, may be determined by any means known in the art. Such methods include statistical-mathematical methods that are implemented in several algorithms that are known to a person skilled in the art, including OptimalCutpoints and maxstat and software programs such as Cutoff Finder, X-tile and Evaluate Cutpoint software. Maxstat is an R-package that performs a test of independence of a response and one or more covariables using maximally selected rank statistics. To analyse whether a cut-off provides a significant discrimination, a significance test, such as a Student's T-Test or Chi-Square Test, can be used.

The number of SVs, for example as determined by the TBL, may be used as an independent prognostic marker in cancer, such as colorectal cancer, especially MSS CRC, and breast cancer. Said cancer is typed as a stage I, II or III cancer, preferably a stage I or II cancer. Patients with a localized cancer, such as a stage I or stage II cancer, but with a high number of SVs such as TBL-high, may be provided with additional therapy such as, for example adjuvant (chemo) therapy after surgery to remove the primary tumor.

Said number of SVs may be determined, for example, by methods described in this application including whole genome sequencing, shallow-whole genome sequencing, exome sequencing such as whole exome sequencing, and targeted genome sequencing.

Apart from the number of SVs, or in addition thereto, SVs that affect exon sequences in MACROD2, were found to be associated with high TBL, while SVs that only affect MACROD2 intronic sequences do not. Hence, an SV that affects exon sequences in MACROD2 can be used as an independent marker for poor prognosis, or can be used in addition to TBL-high. Similarly, mutations in Tumor Protein 53 (TP53) (HGNC: 11998 NCBI Entrez Gene: 7157 Ensembl: ENSG00000141510) can be used in addition to TBL-high, or in addition to TBL-high and presence or absence of an SV that affects exon sequences in MACROD2, to predict tumor prognosis, whereby mutations in TP53, TBL-high and presence of an SV that affects exon sequences in MACROD2, are indicative of a poor prognosis.

After analysis of a potential SV as tumor marker that is specific for a tumor of an individual, said identified tumor marker may be used for minimally-invasive and highly-sensitive monitoring, for example, a response to cancer treatment and recurrence surveillance. The identification of one or more tumor-specific, personalized tumor markers allow detection of tumor cells or of cell-free circulating tumor DNA (ctDNA) in a liquid biopsy of the patient.

For this, the invention provides a method for monitoring tumor progression in a patient, comprising identifying a structural variant (SV) in the patient by performing a method of the invention, providing a first biopsy from the patient, analyzing the first biopsy for presence or abundance of a SV, providing a second biopsy from the patient, whereby the provision of the second biopsy is separated in time from the provision of the first biopsy, analyzing the second biopsy for presence or abundance of the SV, and recording and comparing presence or abundance of the SV in the two biopsies.

Said first, second and, optionally, further biopsies preferably are liquid biopsies, preferably a blood sample.

In between the first, second and/or further biopsies, the patient may be treated by therapy. Said therapy preferably is selected from surgery, chemotherapy, radiation therapy, targeted therapy, immunotherapy, stem cell or bone marrow transplant, hormone therapy, or a combination thereof.

The identification of one or more SVs in specific genes may further have consequences for detection of cancer, whether to apply therapy (prognosis, e.g. based on ‘tumor break load’) and/or the type of therapy that is applied. For example, MACROD2 is hardly affected by SVs in adenomas while highly prevalent in colorectal cancer, and thereby indicative for malignant transformation. In addition, MACROD2 expression is predictive for a poor response to 5FU-based chemotherapy in stage III colon cancer (van den Broek et al., 2018. Oncotarget 9:29445-29452); based on its function in double strand break repair, SVs in MACROD2 are also expected to impact response to radiotherapy. Hence, presence in a sample comprising colorectal tumor cells of a SV in MACROD2 is indicative of presence of a colorectal cancer cells, and may support surgery without radiotherapy. Surgery may be combined with chemotherapy without 5FU. Said chemotherapy may include capecitabine and oxaliplatin, either as such or combined with a vascular endothelial growth factor inhibitor such as bevacizumab, ziv-aflibercept, or ramucirumab, an epidermal growth factor receptor inhibitor such as cetuximab or panitumumab, irinotecan, trifluridine and tipiracil, or a combination thereof.

The presence of SVs in WWOX, GMDS, FHIT and/or PIBF1 is more prevalent in a subgroup of metastatic CRCs with poor prognosis (van den Broek et al., 2015. Plos One 10: e0138141) that is co-clustering with microsatellite instable (MSI) samples. The presence of SVs in WWOX and GMDS is more prevalent in TCGA COADREAD MSI CRCs compared to TCGA COADREAD MSS CRCs (Table 7). This means that SVs in these genes provide a selective advantage to these tumors. Considering these tumors are well recognized by the immune system due to their high Tumor Mutational Burden (TMB), it is likely that SVs in these genes help to evade the immune response. Consequently, MSI tumors with one or more SVs in WWOX FHIT, GMDS and/or PIBF1 are therefore expected to respond well to immunotherapy, including an immune checkpoint inhibitor such a PD1/PDL1 inhibitor and/or a CTLA-4 inhibitor.

Said CTLA4 inhibitor includes ipilimumab (Bristol-Myers-Squibb).

Said PD1/PD-L1 blocker includes antibodies such as atezolizumab (Roche), cemiplimab (Sanofi), pembrolizumab (Merck), nivolumab (Bristol-Myers Squibb), pidilizumab (Medivation/Pfizer), atezolizumab (Roche/Genentech), avelumab (Merck/Serono and Pfizer), durvalumab (AstraZeneca), MEDI0680 (AMP-514; AstraZeneca), spartalizumab (PDR001; Novartis); and BMS-936559 (Bristol-Myers Squibb); fusion proteins such as a PD-L2 Fc fusion protein (AMP-224; GlaxoSmithKline); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl] methyl]piperidine-2-carboxylic acid), BMS202 (PD-1/PD-L1 Inhibitor 2; WO2015034820; N-[2-[[[2-methoxy-6-[(2-methyl [1,1′-biphenyl]-3-yl)methoxy]-3-pyridinyl]methyl]amino] ethyl]-acetamide), and PD-1/PD-L1 Inhibitor 3 (WO/2014/151634; (3S,6S,12S,15S,18S,21S,24S,27S,30R,39S,42S,47aS)-3-((1H-imidazol-5-yl)methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13,). Further anti-PD1 molecules include antibody-drug-conjugates such as ladiratuzumab vedotin (Seattle Genetics). Therefore, the presence or absence of SVs in MACROD2, FHIT and WWOX may impact disease prognosis and treatment regimens.

In addition, a high Tumor Break Load (high TBL), including a high TBL expression phenotype, was found to be associated with poor prognosis. A high TBL may thus warrant treatment including surgery and chemotherapy, even of early stages colorectal cancers. Said chemotherapy may include 5-FU, 5-FU with leucovorin, 5-FU with leucovorin and oxaliplatin, 5-FU with leucovorin and irinotecan, capecitabine with irinotecan, capecitabine with oxaliplatin, either as such or combined with immunotherapy, including an immune checkpoint inhibitor such a PD1/PDL1 inhibitor and/or a CTLA-4 inhibitor.

### 5 EXAMPLES

### Example 1—Clinical and Biological Impact of SVs

### Materials and Methods

Public Data from the Cancer Genome Atlas

For these studies we made use of public data obtained from the cancer genome atlas (TCGA) project, which offers for multiple cancer types access to whole-exome sequencing (WXS) data to determine SNVs, Affymetrix SNP 6.0 array data and/or shallow whole genome sequencing (WGS) data to determine SCNAs, RNA sequencing data to determine gene expression profiles, and pathological and clinical data to refer to, among others, tumor stage and patient survival data (Grossman et al., 2016. N.Engl.J.Med 375:1109-1112).

### MSI Status

Genome instability in cancer can be caused by distinct defects in the DNA repair mechanism. One main distinction to make is between tumors that suffer from microsatellite instability (MSI) and tumors that are microsatellite stable (MSS). For this dataset, MSI status of the adenocarcinomas was evaluated as described (The Cancer Genome Atlas Network, 2012. Nature 487(7407): 330-337). In brief, MSI status was determined using a panel of four mononucleotide repeat sequences (polyadenine tracts BAT25, BAT26, BAT40, and transforming growth factor receptor type II; Kim et al., 2000. Anticancer Res 20:1499-502)) and three dinucleotide repeat sequences (CA repeats in D2S123, D5S346, & D17S250; Fleisher et al., 2000. Cancer Res 60:4864-4868) or the MSI Analysis System, Version 1.2 (Promega, Madison, WI) using five mononucleotide repeat markers (BAT-25, BAT-26, NR-21, NR-24, and MONO-27) and pentanucleotide repeat markers (Penta C and Penta; Belt et al., 2011. European J Cancer 47:1837-1845). Altered size of no marker in tumor DNA resulted in a classification of the tumor as microsatellite-stable (MSS), one or two altered markers (<30%) as low levels of MSI (MSI-L), three altered markers (43%) as equivocal, and five to seven altered markers (>70%) as high levels of MSI (MSI-H).

### SCNA Status

In this study we made use of data obtained from 633 colorectal adenocarcinoma cases, 1098 breast invasive carcinoma cases and 585 lung adenocarcinoma cases described by The Cancer Genome Atlas (The Cancer Genome Atlas Network, 2012. Nature 487(7407): 330-337; The Cancer Genome Atlas Network, 2012. Nature 490(7418): 61-70; The Cancer Genome Atlas Research Network, 2012. Nature 489(7417): 519-525). SNV detection was performed by the NCI Genomic Data Commons (Grossman et al., 2016. N Engl J Med 375:1109-1112). Germline mutations were excluded using a panel of all TCGA blood normal genomes. The Tumor Mutational Burden (TMB) was calculated from the called masked somatic mutation calls using the R package maftools (version 2.4.05). SCNA analysis was performed by the NCI Genomic Data Commons (Grossman et al., 2016. N Engl J Med 375:1109-1112) using Affymetrix SNP 6.0 array data. In brief, SNP 6.0 array data were first normalized for their array intensities and analyzed using Birdsuite to estimate raw copy numbers (Korn et al., 2008. Nat Genet. 40:1253-1260). The raw copy numbers were further normalized using tangent normalization with a panel of normals. The tangent normalized copy numbers were segmented using circular binary segmentation analysis from the DNACopy R-package. Masked copy number segment files were generated by removing the Y chromosome and predefined probe sets from Genomic Data Commons.

### Detection of SCNA-Associated SVs

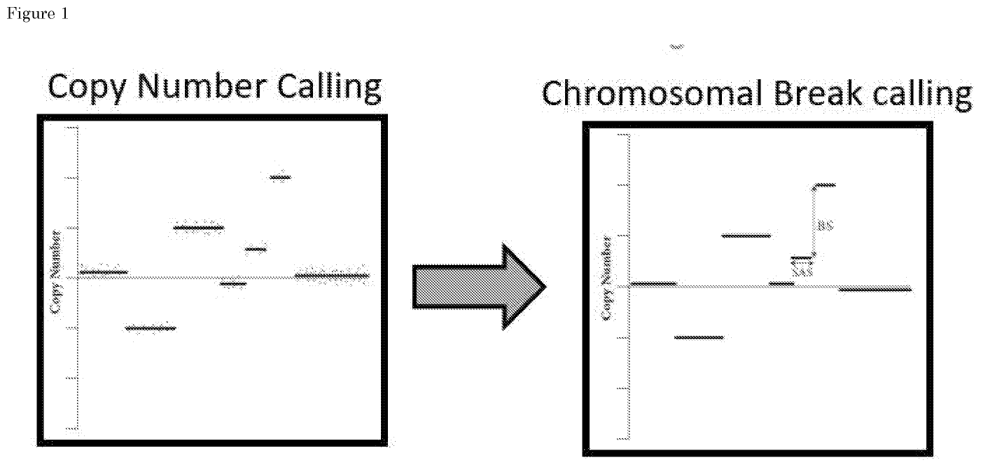

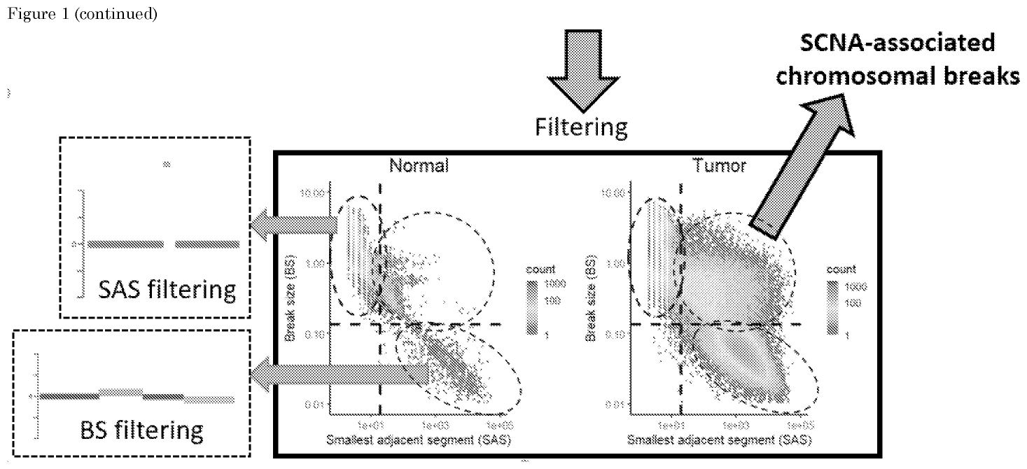

SCNA-associated SVs, i.e. ‘unbalanced’ somatic chromosomal breaks, were determined from masked copy number segment data by filtering, using two parameters (FIG. 1):
1. the smallest adjacent segment size (SAS)
2. the break size (BS).
   The smallest adjacent segment size is the probe count of the smallest segment adjacent to the chromosomal break. The break size is the absolute difference in DNA copy number between the segments adjacent to the chromosomal break. Chromosomal breaks with a SAS<20 or a BS<0.135 were filtered. In this way chromosomal breaks present in normal tissue samples were filtered out from the list of unbalanced somatic chromosomal breaks (FIG. 1).

### Determination of Tumor Break Load (TBL)

The sum of SCNA-associated SVs, i.e. unbalanced somatic chromosomal breaks per tumor sample, is further referred to as the ‘Tumor Break Load’ (TBL).

### Building a TBL Classifier

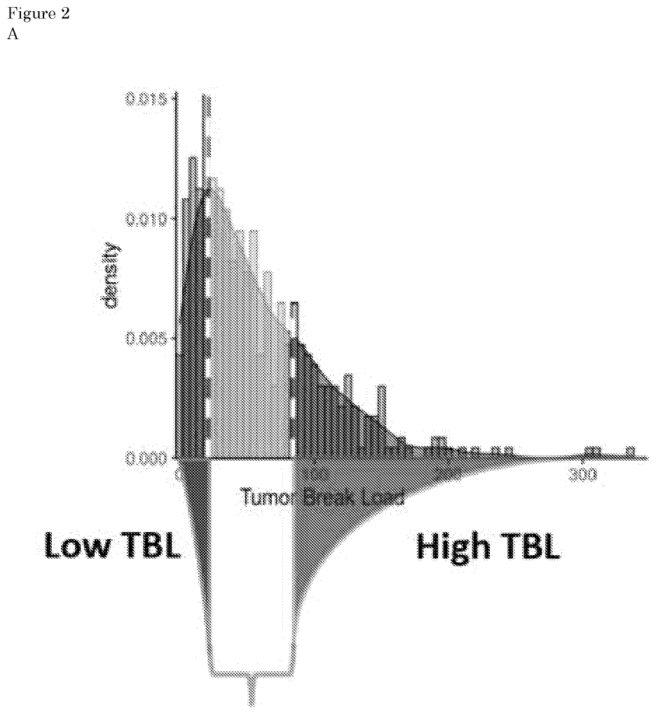

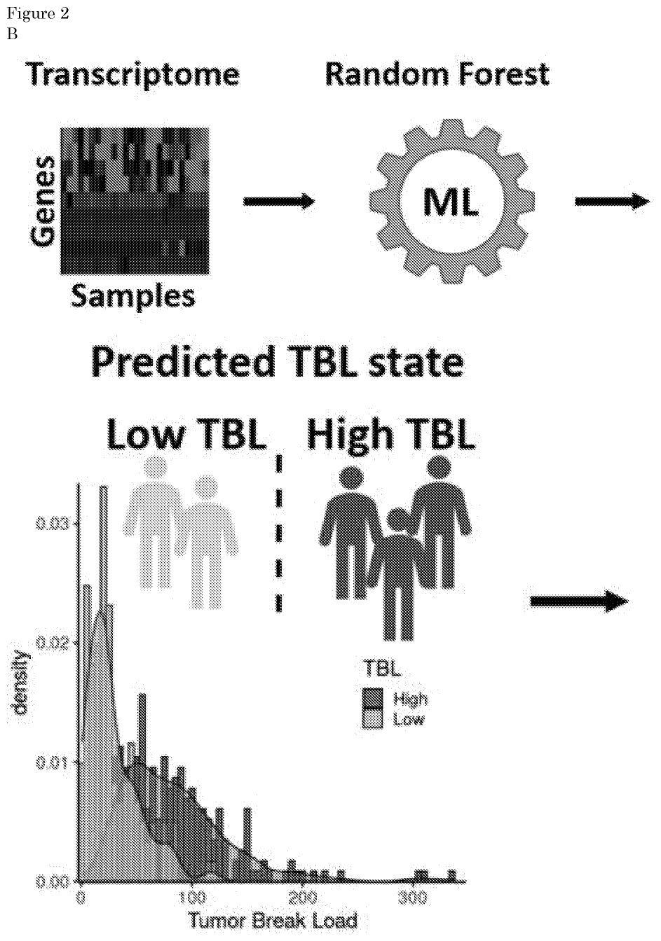

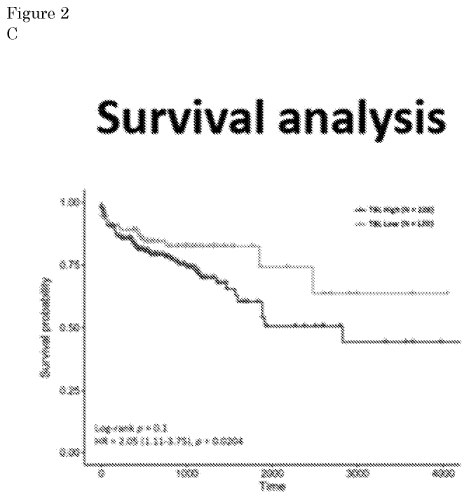

Samples from the upper and lower quantile of the Tumor Break Load (TBL) distribution were selected and denoted in the groups ‘High TBL’ (>75%) and ‘Low TBL’ (<25%). A model was trained to classify the TBL phenotypic behavior of all TCGA (COlon ADenocarcinomas (COAD) and REctal ADenocarcinomas (READ) COADREAD microsatellite stable (MSS) samples, based on RNAseq gene expression data from the same TCGA samples. First, RNA-Seq counts were generated by aligning Illumina reads to the reference genome GRCh38 using a two-pass method with STAR (Dobin et al., 2013. Bioinformatics 29:15-21). Quality assessment of the reads and mapping was performed using FASTQC and Picard Tools. Mapped reads were enumerated using HTSeq-Count (Anders et al., 2015. Bioinformatics 31:166-169) and annotated with GENCODE v22. RNA-Seq counts were further normalized by filtering out zero variance transcripts followed by EdgeR's TMM normalization (edgeR version: 3.28.1; Robinson et al., 2010. Bioinformatics 26:139-140). Next, a Random Forest classifier was implemented using the Classification and Regression Training R package caret (caret version: 6.0.87; Kuhn et al., 2016. caret: Classification and Regression Training. R package version 6.0-71. https://CRAN.R-project.org/package=caret). Normalized gene expression data from the ‘High TBL’ and ‘Low TBL’ groups were used to develop the TBL classification model; this resulted in gene expression-based predictions of a “High-TBL expression phenotype” and “Low-TBL expression phenotype” for all samples. The model was trained using a 65%/35% train-test split with a 10-times 5-fold repeated CV loop for train and feature selection. For feature selection, recursive feature elimination was performed with a 25 step 10-300 feature range using the AUC of the precision recall curve calculated with the R package PRROC (version 1.3.1, Keilwagen et al., 2014. PLOS One 9: e92209) as performance metric and Random Forest as model. Parameter tuning was performed using a 10 value vector for the parameter mtry. The model performance was evaluated with the 35% test set using the AUC of the ROC (receiver operator curve) and the PRC (precision recall curve). The trained model was used to classify the TBL state of all TCGA COADREAD microsatellite stable (MSS) samples (FIG. 2A-C).

### Survival Analysis

Kaplan-Meier survival analysis and Cox proportional-hazard analysis was conducted on the classified patient groups (′High TBL′ and ‘Low TBL’ expression phenotype) to visualize and calculate the significance of differences between the classified ‘High TBL expression phenotype’ and ‘Low TBL expression phenotype’ patients. The Kaplan-Meier survival plots were visualized using the R package survminer (Kassambara et al., 2020. R package version 0.4.8. available at CRAN.R-project.org/package=survminer). Log-rank test P-values and Hazard ratios were calculated using the R package survival (version 3.2.7, Therneau and Grambsch 2000. Modeling Survival Data: Extending the Cox Model. Springer Verlag, New York. ISBN 0-387-98784-3.).

### Determining Biological Impact of Genes Affected by SVs

Similar to the methodology described to build a TBL classifier, the biological impact of genes frequently affected by unbalanced SVs was estimated by creating models to predict the somatic chromosomal break SV status of that gene using gene expression data. The prediction of the (SNV-based) mutational status of APC, TP53 and KRAS was taken along as positive control. For each gene, a model was created to predict the somatic chromosomal break SV status in that gene in a patient. Model performance was further assessed using a permutation test with a model performance distribution of 100 class label permutations. The p-value is calculated by estimating the distribution fraction surpassing the gene performance. This fraction (p-value) indicates how significant the gene model performance is, thereby indicating if having (unbalanced) SVs in that gene has a biological impact.

### Results

### Main Objectives

We previously demonstrated high prevalence and clinical relevance of SVs in colorectal cancer, as described in the introduction. However, our understanding of the clinical utility of SVs and the biological impact of SVs in cancer is lacking. We therefore investigated the following aspects of SVs:
1. The abundancy of SVs in a given tumor genome as a generic variable for genome instability, further referred to as Tumor Break Load (TBL)
2. The impact of SVs in a given gene on tumor biology, in particular gene expression profiles.

### Tumor Break Load (TBL) has Prognostic Value

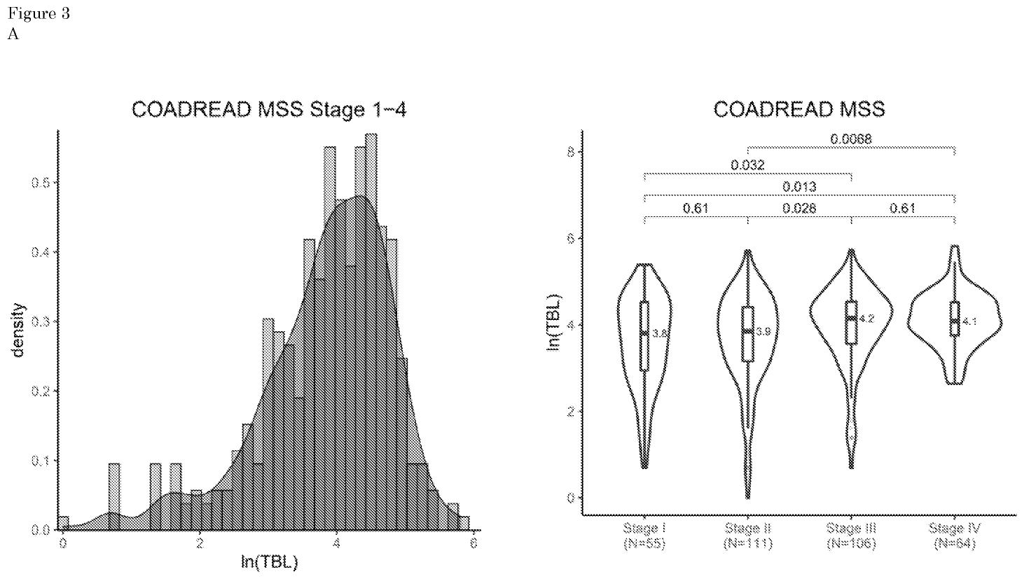

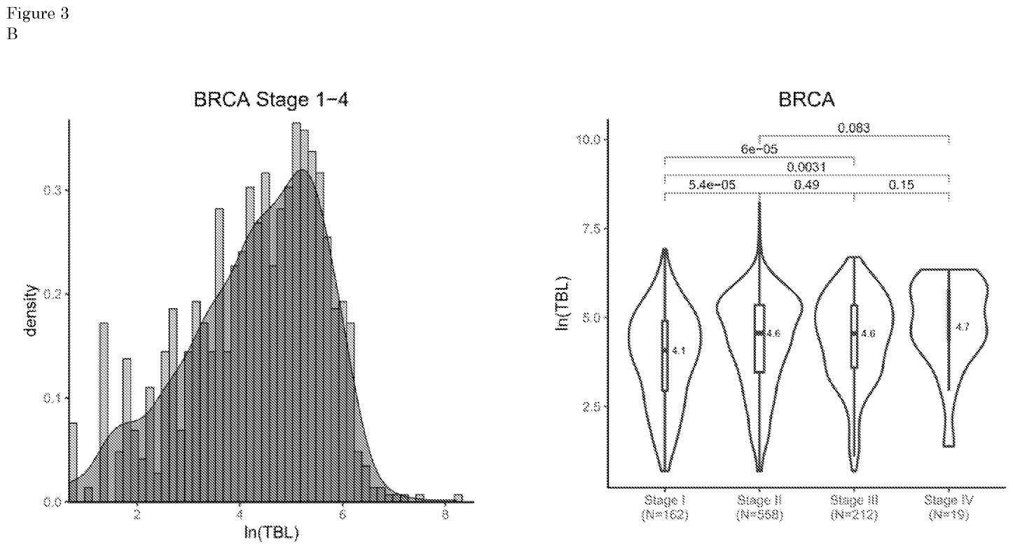

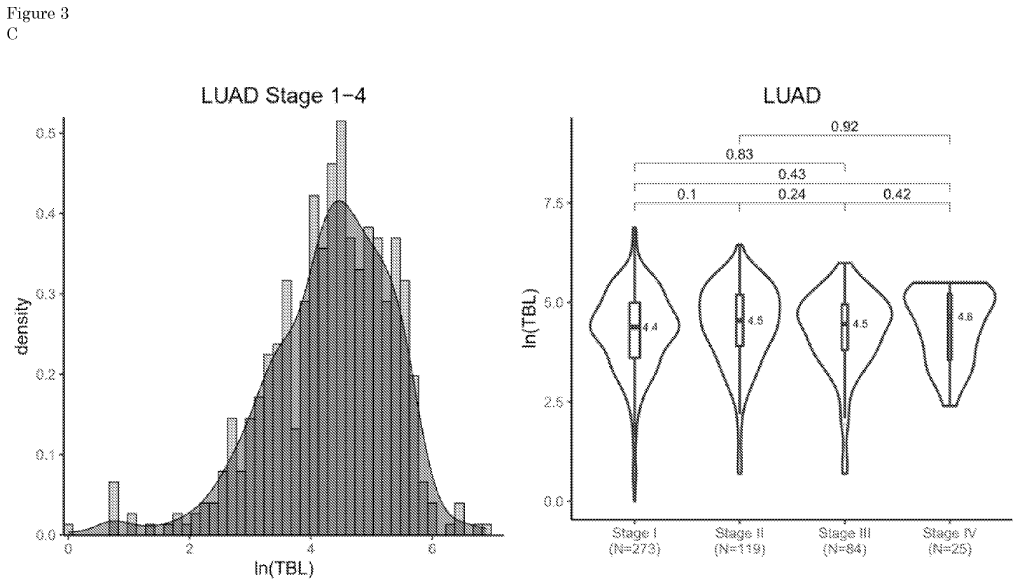

Making use of TCGA data, the distribution of TBL was determined across the series of MSS colon and rectal cancer (COADREAD), breast cancer (BRCA) and lung cancer (LUAD) (FIG. 3A-C, left panels). These data demonstrate the inter-tumor TBL variability within each of these cancer types. To address the question whether TBL is a feature that may be associated with progression to metastatic disease, the TBL distribution was compared per tumor stage in each of these series (FIG. 3A-C, right panels). For MSS colon and rectal cancer samples, a significant increase in TBL was observed in stage III disease compared to stage II disease (FIG. 3A), which marks the transition from localized disease (stage II) to disease that is spreading to the lymph nodes (stage III). The breast cancer series indicated a significant increase in TBL in stage II disease compared to stage I disease (FIG. 3B), which also reflects the transition from localized disease without affected lymph nodes (stage I) to disease that may be spreading to the lymph nodes (stage II). No significant differences in TBL were observed among the various lung cancer stages (FIG. 3C)

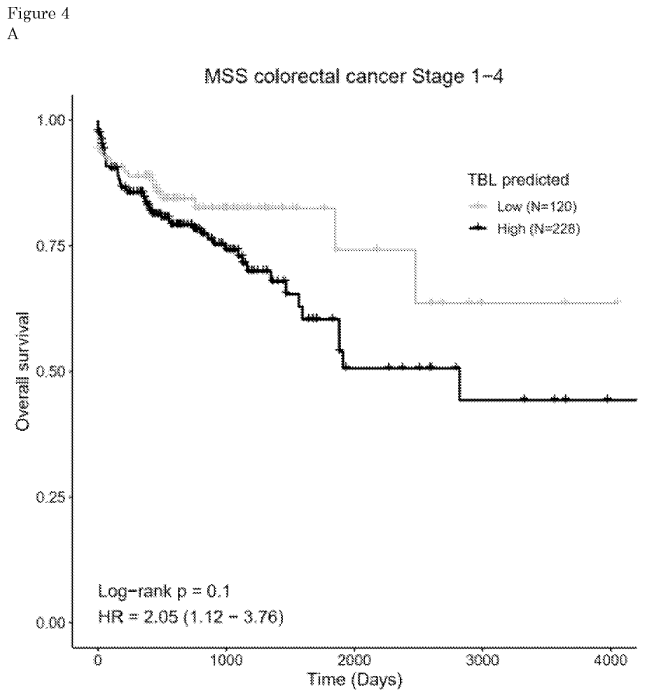

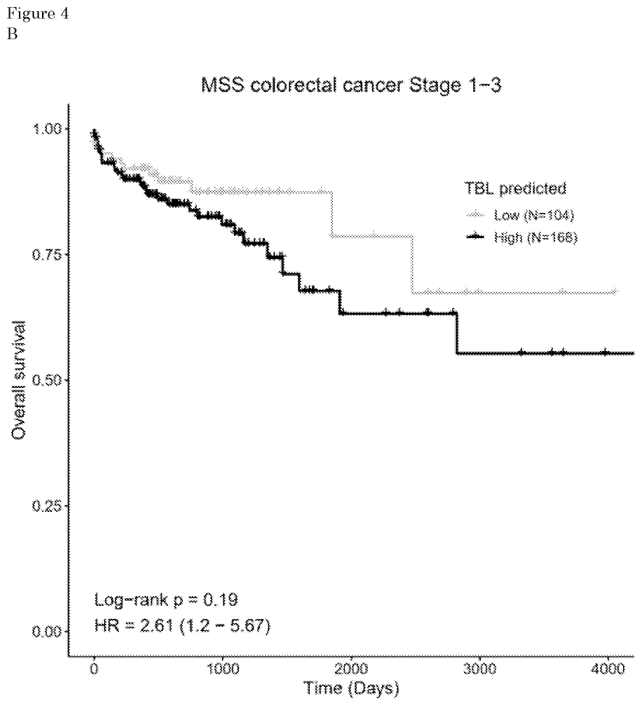

To further explore the putative clinical impact of TBL in CRC, a classifier was constructed to classify each MSS COADREAD cancer in either a ‘High TBL’ or ‘Low TBL’ group (FIG. 2), which was subsequently used for overall survival analysis. Kaplan-Meier curves of TCGA COADREAD MSS patients illustrate that ‘TBL-high’ patients tend to have a worse prognosis than ‘TBL-Low’ patients based on the considerable Hazard Rates, both when analysing cancers from all stages (FIG. 4A) and when focusing on localized disease stage I-III (FIG. 4B). Impact of SVs in a given gene on tumor biology

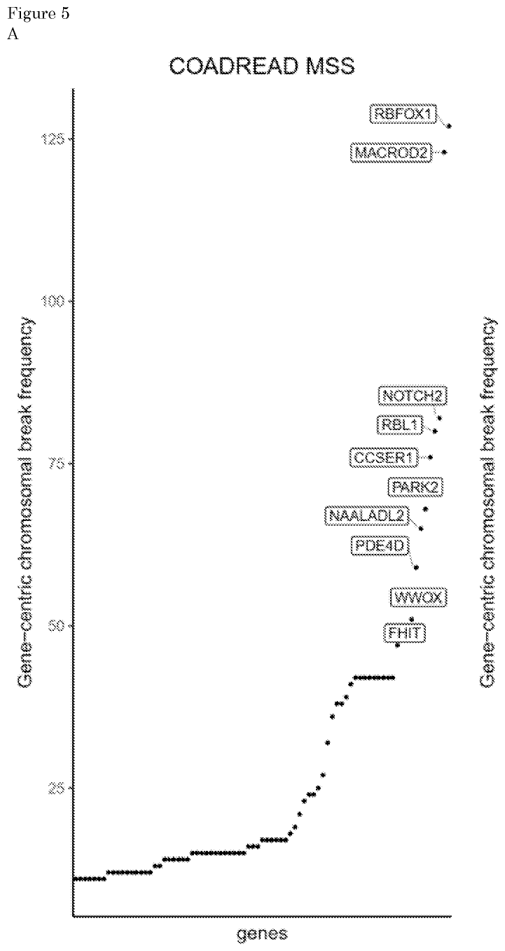

Genome instability is an important feature of cancer. In clinical practice tumor mutation burden (TMB), indicative for the extent of SNVs in the cancer genome, is already used as a generic variable to judge who likely benefits from immunotherapy. At the same time, the exact position of SNVs matters to determine what oncogenes and tumor suppressor genes were mutated and drive tumorigenesis. Conceptually, the genes that are most frequently affected by SNVs such as APC, TP53, and KRAS in case of CRC are most likely to ‘drive’ the process of tumor progression rather than being ‘innocent passenger bystanders’. Likewise, in addition to demonstrating the potential of TBL to be used as a generic variable associated with prognosis, it was examined what genes were most frequently affected by SVs in the TCGA MSS COADREAD, BRCA and LUAD series (FIG. 5). For COADREAD these results largely overlap and therefore verify the results from our previous (array-CGH-based) studies (van den Broek et al., 2016: Oncotarget 7:73876-73887; van den Broek et al., 2015. PLOS One 10: e0138141), with MACROD2 and NOTCH2 among the top hits (FIG. 5A). In breast cancer NOTCH2 is the gene that is most frequently affected by SVs (FIG. 5B) while HCN and PARP8 are most frequently affected by SVs in lung cancer (FIG. 5C).

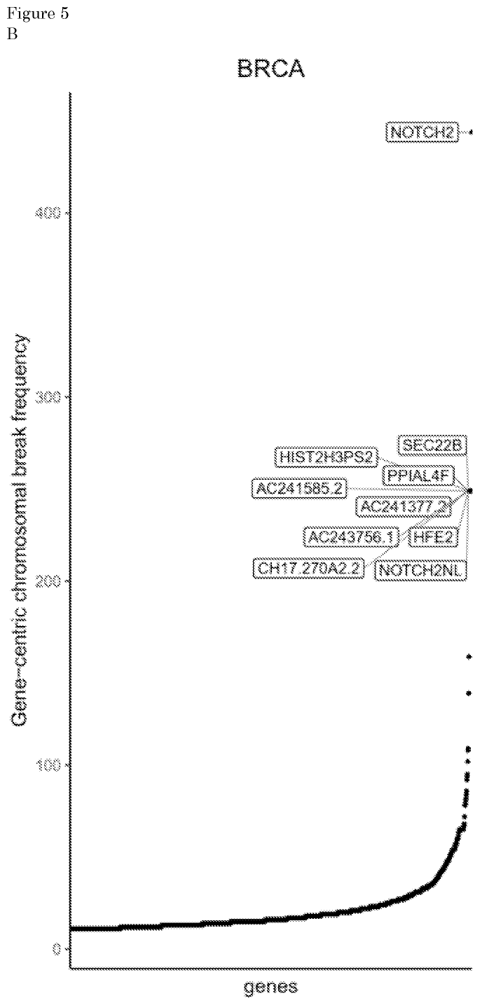

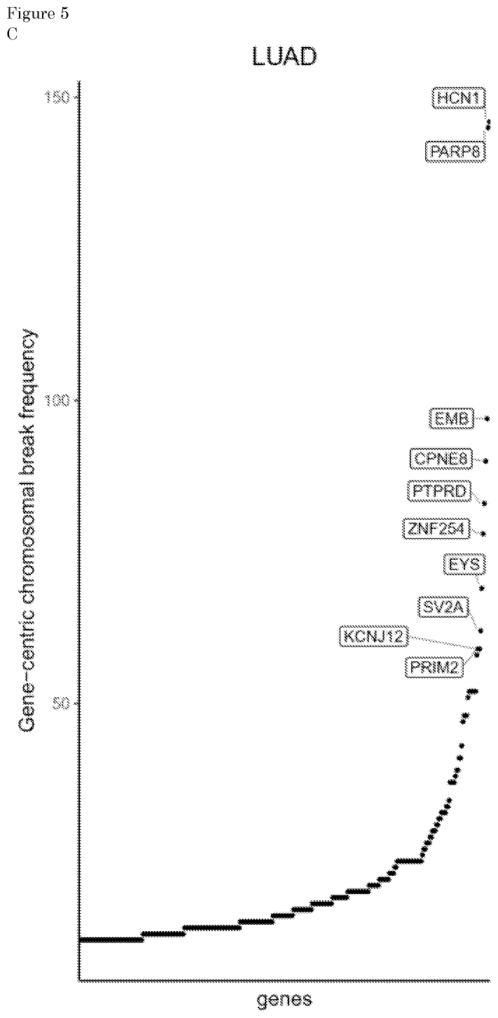

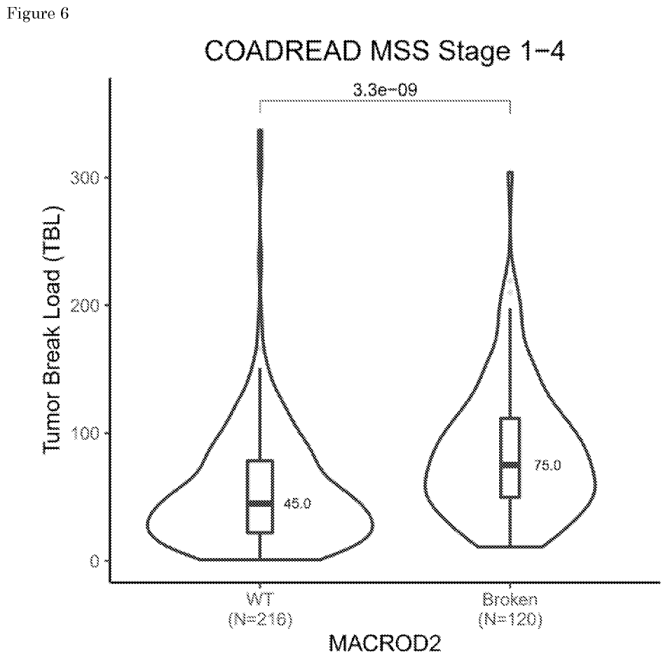

It is of interest to note that both MACROD2 (COADREAD series) and PARP8 (LUAD series) play a role in double strand break (DSB) repair while being predominately affected by double strand breaks (SVs) themselves. To further illustrate this point, we compared the TBL in COADREAD MSS samples in which MACROD2 was not affected by SCNA-associated SVs to the samples in which MACROD2 was affected by SVs. A highly significant difference in TBL was observed (FIG. 6), indicating that SVs in MACROD2 are strongly associated with an increased TBL. These data conform to current literature describing MACROD2 to play a functional role in DNA DSB repair (Sakthianandeswaren et al., 2018. Cancer Discov 8:988-1005) and implying that SVs in MACROD2 may even be causally responsible for DSB repair defects in cancer.

The observation that certain genes may be frequently affected by SVs does not prove that SVs in these genes in fact do affect tumor biology. Moreover, the resolution of mapping SVs to the genome using array-data is insufficient to position SVs with (near) nucleotide resolution onto the genome. To estimate the impact of SVs in a given gene on tumor biology in the TCGA data we hypothesized that SV alterations that do affect tumor biology will reflect this effect in subsequent changes in (genome-wide) mRNA gene expression profiles. Similar to the methodology used to demonstrate that TBL impacts tumor biology (FIG. 2) we now applied machine learning approaches to ‘predict’ the absence or presence of an SV in a given gene in a given tumor sample based on mRNA gene expression data. Using TCGA COADREAD MSS data, models were trained to predict SV status. This analysis was performed on the genes most frequently affected by SVs in TCGA data (FIG. 5A) combined with the genes most frequently affected by SVs in CRCs compared to adenoma precursor lesions (van den Broek, thesis chapter 5, VU University Medical Center 16 Dec. 2016). The AUC for the well-known single nucleotide variation (SNV)-mutated (colorectal) cancer genes APC, KRAS, and TP53 were taken along for comparison as ‘positive control’ reference. The ‘predictability’ was expressed as AUC (based on ROC analysis) and should exceed 0.5 (indicative for ‘random’). As shown in FIG. 7 and Table 1, this method succeeded to predict mutations in APC, TP53 and KRAS with reasonable accuracy, confirming the validity of this approach. Many of the genes that we detected to be commonly affected by SVs exhibited similar predictability, supporting a functional effect on tumor biology of SVs in these genes. The predictability of SVs in NOTCH2 was highest of all, even higher than that of SNVs in KRAS or TP53. Looking across different cancer types it is worth mentioning that NOTCH2 appears to be the gene that is most frequently affected by SVs in the TCGA BRCA series (FIG. 5B). Biologically NOTCH2 is involved in NOTCH signalling, a key signal transduction pathway to balance between stem cell maintenance and proliferation/differentiation and well recognized as one of the key signal transduction pathways in tumor development.

Considering the putative impact of SVs in FHIT and WWOX on prognosis in the metastatic setting (van den Broek et al., 2015. PLOS One 10: e0138141), predictability of SVs in FHIT and WWOX was relatively small (FIG. 7). However, in our previous work SVs in FHIT and WWOX selectively clustered among MSI tumors while we now focused on the analysis of MSS tumors. These data emphasize that further investigation of SVs in MSI tumors is needed.

TABLE 1
Significance of the predictive models, as indicated
by the p-values calculated by a permutation test.

| | | Permutation | |
|---|---|---|---|
| Gene/locus | AUC | AUC | P value |
| APC* | 0.660 | 0.501 ± 0.07 | 0.01 |
| TP53* | 0.835 | 0.488 ± 0.048 | <0.01 |
| KRAS* | 0.821 | 0.487 ± 0.061 | <0.01 |
| RBFOX1 | 0.642 | 0.486 ± 0.057 | <0.01 |
| MACROD2 | 0.647 | 0.501 ± 0.056 | <0.01 |
| Locus 20p11.1* | 0.796 | 0.504 ± 0.056 | <0.01 |
| PARK2; PACRG | 0.732 | 0.492 ± 0.064 | <0.01 |
| NOTCH2 | 0.900 | 0.494 ± 0.071 | <0.01 |
| RBL1 | 0.713 | 0.482 ± 0.068 | <0.01 |
| CCSER1 | 0.640 | 0.494 ± 0.069 | 0.02 |
| NAALADL2 | 0.629 | 0.49 ± 0.073 | 0.04 |
| PDE4D | 0.557 | 0.499 ± 0.075 | 0.25 |
| WWOX | 0.599 | 0.499 ± 0.079 | 0.09 |
| FHIT | 0.521 | 0.486 ± 0.075 | 0.32 |
| AGBL4 | 0.541 | 0.496 ± 0.079 | 0.29 |
| PRKG1 | 0.642 | 0.492 ± 0.079 | 0.02 |
| Locus 1q21.1** | 0.855 | 0.497 ± 0.081 | <0.01 |

### Summarizing Conclusions Example 1

The studies described in example 1 demonstrate:
Tumor Break Load (TBL) is a generic variable that can be used to characterize genome instability features of a given tumor sample. Examples are given for TCGA series of colorectal cancer, breast cancer, and lung cancer.
TBL has prognostic value. Increases in TBL are associated with dissemination of tumor cells to the lymph nodes and beyond in colorectal and breast cancer. The tendency of TBL to be associated with survival is shown for CRC.
The genomic position of SVs in cancer is not random. Many genes are frequently affected by SVs. The distribution of the frequencies in which these genes are affected by SVs differs among cancer types, implying that the biological impact of these SV alterations are to some extent cancer type specific.
In many cases the presence of SVs in or near genes do affect tumor biology, similar to known driver SNVs. One example is SVs in MACROD2, which likely affect the efficiency of double strand break repair in colorectal cancer. Another example is SVs in NOTCH2, which likely plays a key role in (cancer) stem cell biology in colorectal and breast cancer.

### Example 2—the Genomic Location of SVs

### Materials and Methods

Public Data from the Hartwig Medical Foundation

A data set of 464 stage IV CRC biopsies originating from 459 individual patients was provided by the Hartwig Medical Foundation (HMF) (Priestley et al., 2019. Nature 575:210-216). The biopsies were taken from the sites of metastasis. The data set contained clinical data, raw Whole Genome Sequencing (Illumina HiSeq X-Ten) reads of tumor and patient-matched normals, and structural variant calls. The biopsies were collected in 36 different Dutch hospitals as part of the Center for Personalized Cancer Treatment's Drug Rediscovery Protocol (DRUP) trial. The HMF used Manta (Chen et al., 2016. Bioinformatics 32:1220-2) to call structural variants. Since Manta results in quite a high number of false positives, the HMF developed Break Point Inspector (BPI) to increase the accuracy (Priestley et al., 2019. bioRxiv 415133). Matching normal blood samples were used to correct for germline variants. Ensembl GRCh37 (Cunningham et al., 2019. Nucleic Acids Res 47: D745-D751) was used as the reference genome.

### Descriptives

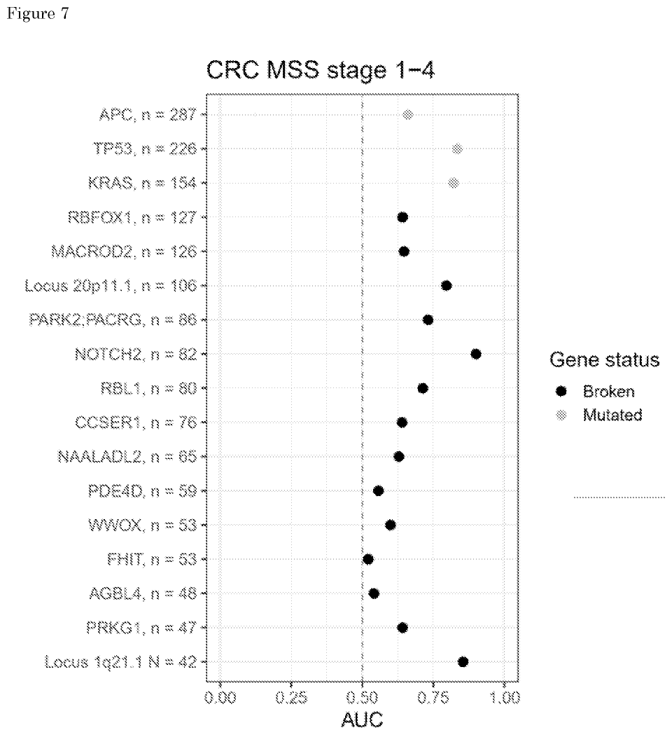

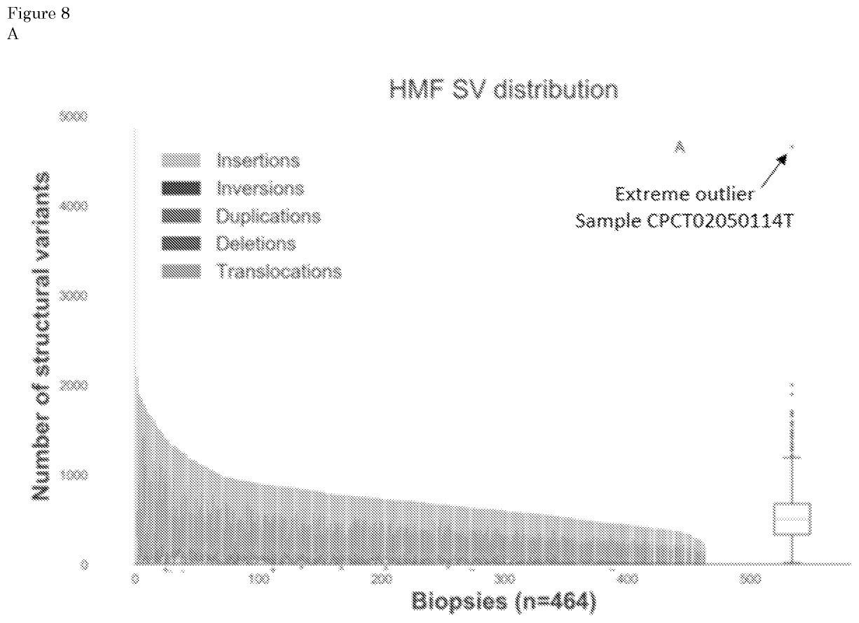

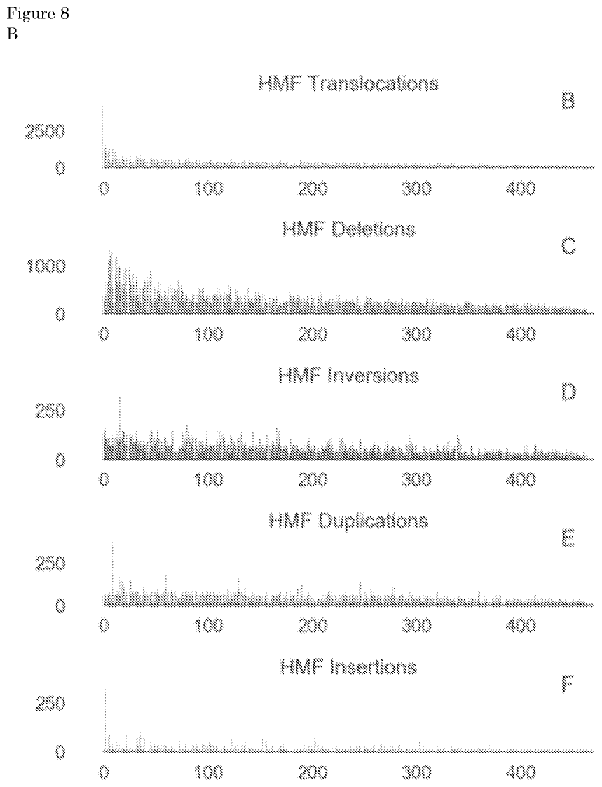

Python V3.7 (Python Software Foundation) and R V3.4.3 (R Core Team) were used to analyze the SV data. First, an overview of the distribution of structural variants throughout the entire data set was created. The Manta algorithm assigned one of five (translocations, deletions, inversions, insertions and duplications) event types to the SVs. Stacked bar plot were created of all types of all samples (FIG. 8). Running BPI results in a call of the start and end coordinate of every SV. These coordinates were used as the chromosomal breakpoints. Deletions, duplications and inversions have two breakpoints, insertions have a single breakpoint, and translocations have one breakpoint on each chromosome. To manually confirm the location of these breakpoints the coordinates of the SV calls made by the HMF were compared with the raw WGS reads in the Integrative Genomics Viewer (IGV) (Thorvaldsdóttir et al., 2017. Briefings Bioinformatics 14:178-192). To this end Browser Extensible Data (BED) files were created of the structural variant calls. After confirming the chromosomal breakpoint coordinates in IGV, an overview was constructed of all the breakpoints partitioned by structural variant type per chromosome. To this end, mirrored histograms with a bin size of 15 kb were created, displaying both the total breakpoints and the number of samples with a breakpoint. Furthermore, BED files were written of the chromosomal breakpoints to be able to visualize the breakpoints across the genome for the entire data set in IGV. The genomic subregions indicated 7.5 kb bins that are statistically more frequently affected by SVs than random. After annotating the breakpoints to genes using the genomic coordinates from Ensembl GRCh37 (also known as hg19) a list of the genes most affected by chromosomal breakpoints was created.

### Structural Variant Statistical Analysis

Since not all genes are of the same length and not all biopsies contain the same number of SVs, it was necessary to perform a statistical analysis to find the genes with a significant number of breakpoints. The R package GeneBreak (van den Broek and van Lieshout, 2017; which is available at github.com/stefvanlieshout/GeneBreak) is typically used to both detect breakpoints based on array-CGH data and to subsequently detect recurrent breakpoint regions across samples. Previous breakpoint analysis on CAIRO and CAIRO2 array-CGH data using GeneBreak resulted in a collection of genes with recurrent breakpoints (van den Broek et al., 2015. PLOS One 10: e0138141). Although the HMF data has both a different biological (DNA mutation analysis of metastasis versus primary tumor) and technical (WGS compared to ArrayCGH) origin, the statistical objective remains the same. Therefore, the statistical approach used here was based upon the gene-centric approach from GeneBreak which detects genes with recurrent breakpoints. This statistical approach was implemented in R. In this approach a separate null probability for a break to occur was calculated for every sample and every gene, taking gene length and breakpoint frequency per sample into account. GeneBreak was originally written for ArrayCGH data, using the number of ArrayCGH probes annotated to a gene as an indicator for gene length. Since WGS does not use probes, gene length was inferred directly from Ensembl GRCh37 (hg19).

After running the modified version of GeneBreak it was noticed that some large genes which were expected to show up as being significantly affected by chromosomal breakpoints did not show up as significant in the analysis. In the modified version of GeneBreak, very large genes require a large number of breakpoints to be significant. It was hypothesized that while these large genes might not contain enough breakpoints in total to be significant, they could contain recurrent breakpoints within subregions of the gene. Besides being capable of finding genes that are significantly affected by SVs, GeneBreak can also detect significance on probe level. This feature was utilized by binning the breakpoints into overlapping bins of 15 kb in size (sliding window with 7.5 kb overlap) followed by running GeneBreak on the probe level and a minimum pooling step. The significant 7.5 kb bins were annotated to Ensembl GRCh37 with the aim to find significant subregions within large genes. The genes found by using the gene level approach were compared with the regions found by this simulated probe approach and previous knowledge of genes recurrently affected by SVs.

Data Cleaning—Deleting One Outlier Sample with Extremely Many SVs

Of the original 464 biopsies one sample (CPCT02050114TI) was removed from the analysis. It was considered to be an outlier (see herein below; FIG. 8) which may put too much weight on further breakpoint analysis. There were five individual patients of whom two biopsies were taken. A clustering step showed that these samples do not cluster together, therefore it was decided to leave these samples in. The MSI status of the patients was known.

Data Cleaning—Deleting SV Calls Recurrently Occurring in More than 4 Samples

When examining small genomic regions containing many breakpoints in IGV, it was noticed that in these regions breakpoints very often get called on a single nucleotide. This forms a long vertical line of breakpoints across samples and is thus easily spotted in IGV. When examining the raw reads upon which these calls were based, certain problems with these calls were noticed. First, these locations often have a drop in coverage both in the tumor and normal samples. Furthermore, there is often no difference in appearance between the tumor and the normal. To examine how frequent breakpoints were called at single nucleotide locations the number of breakpoint locations were plotted against the number of samples with an identical breakpoint. The distribution shows that it is quite common in this data set that two, three or four samples share a unique nucleotide as a breakpoint location. In addition, there is a long tail of events called in a larger number of samples with one occurrence of a single breakpoint location being called as a breakpoint in 449 tumor samples (out of a total of 463 tumor samples). All calls from the HMF data set that were shared by more than four tumor samples were removed for further analyses. This enticed a total of 11.067 breakpoint locations and a removed total of 129.934 breakpoint calls. Application of this threshold filter improved the reliability of the data.

### Results

### Main Objectives

The research described in example 1 is based on SNP-array TCGA data. Also in our previous work we made use of array-based (array-Comparative Genomic Hybridization) data to detect SCNA-associated SVs, i.e. SVs that one can detect based on unbalanced chromosomal alterations (van den Broek, thesis chapter 5, VU University Medical Center 16 Dec. 2016; van den Broek et al., 2016: Oncotarget 7:73876-73887; van den Broek et al., 2015. PLOS One 10: e0138141). The probes that were used in the previously described Array-CGH profiling experiments were spaced apart from each other on the genome at approximately 15 kb to 20 kb. Consequently, the resolution at which chromosomal breakpoints could be positioned in the genome using Array-CGH data is at least 15 kb, and probably approximately 50 kb, pending accuracy of the DNA copy number calling. More recently, using Illumina sequencing, whole genome sequencing data of (colorectal cancer) metastases became available from the Hartwig Medical Foundation (HMF; ˜90× coverage of tumor tissue, 30× coverage of matched normal DNA), and algorithms were applied that allowed SV calling at nucleotide resolution (Priestley et al., 2019. Nature 575:210-216). While SCNA-associated SVs are per definition associated with ‘unbalanced’ DNA copy number alterations, the HMF data allowed to also detect ‘balanced’ DNA copy number neutral SV events. With these data in hand, focusing on CRC, we aimed to investigate:
1. The genomic position of SVs in CRC samples
2. The genomic regions in CRC that are enriched for SV events

### General Descriptives

An overview of the distribution of structural variants throughout the HMF data set was created (FIG. 8). On average there are 763 SVs per biopsy with a median of 702 SVs and a total of 354021 SVs. One sample (CPCT02050114T) has a total of 4866 SVs and was considered an outlier and left out of subsequent analyses. Translocations (152746 SVs) and deletions (133995 SVs) form the largest categories of structural variants, respectively. The combined number of inversions, insertions and duplications forms 19% of the total number of SVs. The locations of chromosomal breaks were deduced from the SV calls as described in the Methods section. This resulted in a total of 538754 breakpoints (excluding sample CPCT02050114TI). The precise location where a breakpoint may be determined by Manta and BPI.

To characterize how these breakpoints are distributed throughout the genome all breakpoints of all tumor samples were visualized in IGV. In such plots, every individual ‘tick’ indicates a single breakpoint in a single sample, and denser areas are caused by many breakpoints in multiple samples in close vicinity of each other. It was often found that breakpoints seem to aggregate in a particular region in comparison with the surrounding genome.

Genes that are Frequently Affected by SVs

The first objective was to find the genes that are frequently affected by SVs. Therefore, after removal of sample CPCT02050114T and the removal of exactly identical calls shared by more than four samples (see the data cleaning section in Materials and Methods) the breakpoints called by the HMF were annotated to genes from Ensembl GRCh37 (hg19). Genes that are affected by SVs are listed in Table 2. In total, SVs were detected in 15, 105 different genes. Of these, 979 genes appeared to be affected by SVs in >5% of tumor samples, of which 303 in >10% of samples, of which 55 in >20% of samples, of which 9 in more than 50% of samples. The gene that is most frequently affected by SVs is MACROD2, which appears to be affected in 342 out of 463 metastatic CRC tissue samples (˜74%). These data clearly illustrate that SVs are highly prevalent, e.g. when compared to the prevalence of SNVs in colorectal cancer (The Cancer Genome Atlas Network, 2012. Nature 487(7407): 330-337). Table 2 shows an overview of the top 100 genes that are most frequently affected by SCs in CRC within the HMF series of samples. Please note that all these genes are fairly large. Where the average gene length is around 87.000 base pairs (Cunningham et al., 2019. Nucleic Acids Res 47: D745-D751), these significantly broken genes are all a lot larger. These data illustrate the need for a statistical approach to find the genomic regions most frequently affected by SVs independent of genes and gene length.

TABLE 2
Overview of the hundred genes that are most frequently affected
by SVs. Shown are the total number of breakpoints, the
total number and percentage of affected samples (out of
n = 463) and the length of genes in basepairs (bp).

| | | Total | Affected | | |
|---|---|---|---|---|---|
| | | break- | samples | Percentage | Length |
| Rank | Gene | points | (n = 463) | affected | (bp) |
| 1 | MACROD2 | 3353 | 342 | 73.87 | 2057827 |
| 2 | FHIT | 2226 | 332 | 71.71 | 1502097 |
| 3 | RBFOX1 | 2014 | 302 | 65.23 | 1694245 |
| 4 | TTC28 | 1216 | 298 | 64.36 | 701849 |
| 5 | PTPRN2 | 1045 | 281 | 60.69 | 1048730 |
| 6 | PARK2 | 1624 | 276 | 59.61 | 1380351 |
| 7 | CCSER1 | 1078 | 244 | 52.70 | 1474378 |
| 8 | ZDHHC11 | 504 | 236 | 50.97 | 140630 |
| 9 | NAALADL2 | 977 | 235 | 50.76 | 1367065 |
| 10 | WWOX | 957 | 221 | 47.73 | 1113254 |
| 11 | PDE4D | 767 | 199 | 42.98 | 1553082 |
| 12 | PRKG1 | 848 | 198 | 42.76 | 1307165 |
| 13 | IMMP2L | 778 | 195 | 42.12 | 899463 |
| 14 | PRR4 | 446 | 174 | 37.58 | 346653 |
| 15 | AGBL4 | 487 | 159 | 34.34 | 1491058 |
| 16 | LINC00969 | 270 | 156 | 33.69 | 75489 |
| 17 | TYW1B | 262 | 155 | 33.48 | 275048 |
| 18 | SNX29P2 | 233 | 152 | 32.83 | 256988 |
| 19 | CDH4 | 337 | 144 | 31.10 | 688191 |
| 20 | DPP6 | 293 | 143 | 30.89 | 1101813 |
| 21 | EYS | 329 | 142 | 30.67 | 1987242 |
| 22 | UPK3B | 210 | 142 | 30.67 | 508595 |
| 23 | CNTNAP2 | 394 | 141 | 30.45 | 2304637 |
| 24 | ZDHHC11B | 164 | 129 | 27.86 | 56592 |
| 25 | TAS2R14 | 302 | 127 | 27.43 | 234167 |
| 26 | TYW1 | 213 | 126 | 27.21 | 244341 |
| 27 | LRP1B | 350 | 124 | 26.78 | 1900278 |
| 28 | PTPRD | 331 | 123 | 26.57 | 2298477 |
| 29 | MACROD2-AS1 | 196 | 123 | 26.57 | 45262 |
| 30 | DHRS4-AS1 | 153 | 120 | 25.92 | 50108 |
| 31 | DHRS4L2 | 148 | 117 | 25.27 | 36469 |
| 32 | DIP2C | 330 | 115 | 24.84 | 415553 |
| 33 | PRMT8 | 259 | 114 | 24.62 | 212624 |
| 34 | TPTE2P6 | 162 | 114 | 24.62 | 189519 |
| 35 | PCDH15 | 207 | 110 | 23.76 | 1825171 |
| 36 | RPH3AL | 267 | 109 | 23.54 | 173752 |
| 37 | ADARB2 | 246 | 109 | 23.54 | 551597 |
| 38 | GPC5 | 262 | 108 | 23.33 | 1468561 |
| 39 | AVL9 | 176 | 108 | 23.33 | 543478 |
| 40 | LSAMP | 337 | 107 | 23.11 | 2194860 |
| 41 | ROBO2 | 289 | 107 | 23.11 | 1743269 |
| 42 | MUC20 | 143 | 103 | 22.25 | 20241 |
| 43 | ZNF813 | 119 | 103 | 22.25 | 35961 |
| 44 | DLG2 | 250 | 102 | 22.03 | 2172911 |
| 45 | CSMD1 | 194 | 101 | 21.81 | 2059619 |
| 46 | GPC6 | 189 | 98 | 21.17 | 1180560 |
| 47 | LRBA | 178 | 98 | 21.17 | 751285 |
| 48 | CSMD3 | 224 | 97 | 20.95 | 1214171 |
| 49 | CTNNA3 | 224 | 97 | 20.95 | 1783651 |
| 50 | VPS13B | 176 | 97 | 20.95 | 864314 |
| 51 | FCGR3A | 129 | 96 | 20.73 | 89368 |
| 52 | GML | 188 | 95 | 20.52 | 82259 |
| 53 | DIAPH2 | 200 | 94 | 20.30 | 920334 |
| 54 | MAD1L1 | 177 | 94 | 20.30 | 417449 |
| 55 | FCGR2B | 104 | 93 | 20.09 | 97343 |
| 56 | LMF1 | 222 | 92 | 19.87 | 127684 |
| 57 | SMYD3 | 201 | 92 | 19.87 | 757972 |
| 58 | SUMF1 | 186 | 91 | 19.65 | 766467 |
| 59 | TPPP | 108 | 91 | 19.65 | 32627 |
| 60 | CYP11B2 | 99 | 89 | 19.22 | 7284 |
| 61 | MUC4 | 194 | 88 | 19.01 | 65512 |
| 62 | PACRG | 156 | 88 | 19.01 | 588360 |
| 63 | CYP11B1 | 98 | 88 | 19.01 | 7490 |
| 64 | MAN1B1 | 198 | 87 | 18.79 | 22256 |
| 65 | TRAPPC9 | 187 | 87 | 18.79 | 726092 |
| 66 | SGCZ | 143 | 87 | 18.79 | 1148475 |
| 67 | TPO | 198 | 86 | 18.57 | 169488 |
| 68 | MAGI2 | 177 | 85 | 18.36 | 1436497 |
| 69 | HEATR4 | 118 | 85 | 18.36 | 80462 |
| 70 | TRIM16 | 98 | 85 | 18.36 | 56351 |
| 71 | TRIM16L | 95 | 85 | 18.36 | 38120 |
| 72 | SNTG1 | 175 | 84 | 18.14 | 884329 |
| 73 | SDK1 | 164 | 84 | 18.14 | 967552 |
| 74 | PARD3B | 194 | 83 | 17.93 | 1074370 |
| 75 | DTX2P1 | 116 | 83 | 17.93 | 65158 |
| 76 | DTX2P1- | 116 | 83 | 17.93 | 62826 |
| | UPK3BP1- | | | | |
| | PMS2P11 | | | | |
| 77 | CROCC | 106 | 83 | 17.93 | 232706 |
| 78 | KIR3DL1 | 193 | 81 | 17.49 | 142479 |
| 79 | NRXN3 | 161 | 81 | 17.49 | 1622028 |
| 80 | GYPB | 136 | 81 | 17.49 | 144587 |
| 81 | BANP | 107 | 81 | 17.49 | 128074 |
| 82 | LINC00669 | 222 | 80 | 17.28 | 593394 |
| 83 | TDG | 129 | 80 | 17.28 | 23070 |
| 84 | MYLK | 369 | 79 | 17.06 | 274282 |
| 85 | LRRTM4 | 225 | 79 | 17.06 | 845600 |
| 86 | ACTR3B | 89 | 79 | 17.06 | 95629 |
| 87 | GUSBP1 | 98 | 78 | 16.85 | 247539 |
| 88 | DMD | 152 | 77 | 16.63 | 2241764 |
| 89 | GRID2 | 174 | 76 | 16.41 | 1470157 |
| 90 | RASA3 | 171 | 76 | 16.41 | 150892 |
| 91 | MCF2L | 161 | 76 | 16.41 | 205361 |
| 92 | ERBB4 | 143 | 76 | 16.41 | 1163119 |
| 93 | DNM1P47 | 130 | 76 | 16.41 | 43270 |
| 94 | CDH13 | 129 | 76 | 16.41 | 1169796 |
| 95 | CSF2RA | 165 | 75 | 16.20 | 41581 |
| 96 | FAM19A5 | 147 | 75 | 16.20 | 361452 |
| 97 | ZNF761 | 89 | 75 | 16.20 | 26288 |
| 98 | TPM3P9 | 87 | 75 | 16.20 | 12686 |
| 99 | GMDS | 186 | 74 | 15.98 | 621885 |
| 100 | CADM2 | 173 | 74 | 15.98 | 1115447 |

Genomic Subregions that are Frequently Affected by SVs

Following the gene-centric approach to categorize genes according to SV frequencies, the second objective was a gene-independent genome-wide approach to determine what genomic subregions are significantly affected by SVs. The chromosomal breakpoints were combined (‘pooled’) in overlapping bins of 15 kb (sliding window with 7.5 kb overlap) and the probe level statistics from GeneBreak was applied to these bins. Bins were annotated to genes using Ensembl GRCh37 (hg19). Performing genome-wide statistics resulted in 9,069 significantly affected genomic regions of 7.5 kb overlapping with 3695 genes. The top 100 most significantly affected bins are displayed in Table 3. It was noticed that some bins were highly significant but not annotated to any known gene. Therefore, these bins were also annotated to a database of non-coding DNA (Volders et al., 2018. Nucleic Acids Res 47: D135-D139). Cases were found where the breakpoints co-located with these noncoding DNA regions. Table 3 displays the genes with the most significant bins.

TABLE 3
Overview of the top hundred 7.5 kb bins that are most frequently affected by SVs. Shown are the
chromosomal positions, total number of breakpoints, the total number and percentage of affected
samples (out of n = 463), the P-values and False Discovery Rates (FDR), and the genomic position
within known genes. Ranking is based on the P-value and the number of number of samples affected.

| | | | | Nr. of | Nr. of | | | |
|---|---|---|---|---|---|---|---|---|
| Rank | Chromosome | Start | End | breakpoints | samples | P-value | FDR | Gene |
| 1 | 22 | 29062500 | 29070000 | 1004 | 276 | 0 | 0 | TTC28 |
| 2 | 23 | 11730000 | 11737500 | 377 | 115 | 7.04E−217 | 1.44E−211 | |
| 3 | 14 | 59220000 | 59227500 | 535 | 105 | 2.25E−193 | 3.06E−188 | |
| 4 | 12 | 3607500 | 3615000 | 206 | 96 | 1.09E−172 | 1.11E−167 | PRMT8 |
| 5 | 7 | 57442500 | 57450000 | 111 | 92 | 1.19E−163 | 9.76E−159 | |
| 6 | 8 | 143955000 | 143962500 | 98 | 88 | 1.05E−154 | 7.16E−150 | GML, |
| | | | | | | | | CYP11B1 |
| 8 | 12 | 132060000 | 132067500 | 204 | 87 | 1.75E−152 | 8.92E−148 | |
| 7 | 9 | 139995000 | 140002500 | 163 | 87 | 1.75E−152 | 8.92E−148 | MAN1B1 |
| 9 | 6 | 170482500 | 170490000 | 205 | 86 | 2.86E−150 | 1.30E−145 | |
| 10 | 5 | 742500 | 750000 | 93 | 85 | 4.62E−148 | 1.89E−143 | ZDHHC11, |
| | | | | | | | | ZDHHC11B |
| 11 | 14 | 106050000 | 106057500 | 96 | 76 | 1.74E−128 | 6.46E−124 | IGHA2 |
| 12 | 14 | 106852500 | 106860000 | 78 | 75 | 2.41E−126 | 8.19E−122 | IGHV3-37 |
| 13 | 6 | 169087500 | 169095000 | 235 | 71 | 7.48E−118 | 2.35E−113 | |
| 14 | 7 | 72022500 | 72030000 | 90 | 69 | 1.19E−113 | 3.48E−109 | TYW1B |
| 15 | 14 | 24480000 | 24487500 | 75 | 68 | 1.47E−111 | 3.99E−107 | |
| 17 | 7 | 66720000 | 66727500 | 85 | 67 | 1.77E−109 | 4.26E−105 | |
| 16 | 5 | 690000 | 697500 | 71 | 67 | 1.77E−109 | 4.26E−105 | TPPP |
| 18 | 2 | 87937500 | 87945000 | 66 | 66 | 2.10E−107 | 4.78E−103 | |
| 19 | 1 | 119400000 | 119407500 | 91 | 65 | 2.45E−105 | 5.28E−101 | |
| 20 | 7 | 57075000 | 57082500 | 65 | 64 | 2.81E−103 | 5.74E−99 | TNRC18P3, |
| | | | | | | | | SLC29A4P1 |
| 21 | 12 | 11077500 | 11085000 | 70 | 63 | 3.16E−101 | 6.14E−97 | PRR4, PRH2 |
| 22 | 7 | 158130000 | 158137500 | 101 | 62 | 3.48E−99 | 6.47E−95 | PTPRN2 |
| 24 | 20 | 60517500 | 60525000 | 128 | 61 | 3.77E−97 | 6.42E−93 | |
| 23 | 12 | 9555000 | 9562500 | 83 | 61 | 3.77E−97 | 6.42E−93 | |
| 28 | 7 | 158122500 | 158130000 | 100 | 60 | 4.01E−95 | 5.64E−91 | PTPRN2 |
| 27 | 7 | 149730000 | 149737500 | 93 | 60 | 4.01E−95 | 5.64E−91 | |
| 26 | 3 | 195337500 | 195345000 | 70 | 60 | 4.01E−95 | 5.64E−91 | |
| 25 | 1 | 1657500 | 1665000 | 65 | 60 | 4.01E−95 | 5.64E−91 | SLC35E2 |
| 29 | 16 | 29325000 | 29332500 | 62 | 60 | 4.01E−95 | 5.64E−91 | SNX29P2 |
| 30 | 1 | 83850000 | 83857500 | 69 | 59 | 4.17E−93 | 5.33E−89 | |
| 32 | 7 | 142125000 | 142132500 | 66 | 59 | 4.17E−93 | 5.33E−89 | TRBV5-6 |
| 31 | 7 | 76125000 | 76132500 | 65 | 59 | 4.17E−93 | 5.33E−89 | DTX2 |
| 33 | 3 | 123592500 | 123600000 | 222 | 57 | 4.26E−89 | 4.98E−85 | MYLK |
| 34 | 3 | 195345000 | 195352500 | 79 | 57 | 4.26E−89 | 4.98E−85 | |
| 35 | 5 | 780000 | 787500 | 59 | 57 | 4.26E−89 | 4.98E−85 | ZDHHC11 |
| 36 | 14 | 24442500 | 24450000 | 61 | 56 | 4.18E−87 | 4.75E−83 | DHRS4- |
| | | | | | | | | AS1, |
| | | | | | | | | DHRS4L2 |
| 41 | 17 | 20467500 | 20475000 | 60 | 55 | 4.02E−85 | 4.00E−81 | COTL1P2 |
| 39 | 12 | 132150000 | 132157500 | 59 | 55 | 4.02E−85 | 4.00E−81 | RPS6P21 |
| 38 | 7 | 56610000 | 56617500 | 58 | 55 | 4.02E−85 | 4.00E−81 | |
| 37 | 3 | 125685000 | 125692500 | 56 | 55 | 4.02E−85 | 4.00E−81 | ROPN1B |
| 40 | 14 | 74002500 | 74010000 | 56 | 55 | 4.02E−85 | 4.00E−81 | HEATR4, |
| | | | | | | | | ACOT1, |
| | | | | | | | | NT5CP2 |
| 44 | 22 | 25065000 | 25072500 | 69 | 54 | 3.78E−83 | 3.51E−79 | |
| 43 | 8 | 143992500 | 144000000 | 58 | 54 | 3.78E−83 | 3.51E−79 | GML, |
| | | | | | | | | CYP11B2 |
| 42 | 7 | 142140000 | 142147500 | 57 | 54 | 3.78E−83 | 3.51E−79 | TRBV6-7 |
| 45 | 3 | 195450000 | 195457500 | 82 | 52 | 3.14E−79 | 2.67E−75 | LINC00969, |
| | | | | | | | | MUC20 |
| 46 | 12 | 31267500 | 31275000 | 73 | 52 | 3.14E−79 | 2.67E−75 | |
| 48 | 22 | 42517500 | 42525000 | 57 | 52 | 3.14E−79 | 2.67E−75 | NDUFA6- |
| | | | | | | | | AS1, |
| | | | | | | | | CYP2D6 |
| 47 | 21 | 43492500 | 43500000 | 56 | 52 | 3.14E−79 | 2.67E−75 | UMODL1 |
| 49 | 1 | 201225000 | 201232500 | 98 | 51 | 2.77E−77 | 2.22E−73 | |
| 51 | 19 | 56280000 | 56287500 | 55 | 51 | 2.77E−77 | 2.22E−73 | RFPL4AL1 |
| 50 | 14 | 106170000 | 106177500 | 53 | 51 | 2.77E−77 | 2.22E−73 | IGHA1 |
| 55 | 7 | 142470000 | 142477500 | 54 | 50 | 2.39E−75 | 1.77E−71 | PRSS3P1 |
| 52 | 5 | 787500 | 795000 | 51 | 50 | 2.39E−75 | 1.77E−71 | ZDHHC11 |
| 53 | 7 | 6855000 | 6862500 | 50 | 50 | 2.39E−75 | 1.77E−71 | CCZ1B |
| 54 | 7 | 74490000 | 74497500 | 50 | 50 | 2.39E−75 | 1.77E−71 | WBSCR16 |
| 57 | 3 | 123585000 | 123592500 | 118 | 49 | 2.01E−73 | 1.42E−69 | MYLK |
| 56 | 2 | 1597500 | 1605000 | 108 | 49 | 2.01E−73 | 1.42E−69 | |
| 58 | 4 | 9480000 | 9487500 | 58 | 49 | 2.01E−73 | 1.42E−69 | OR7E85P |
| 63 | 19 | 2347500 | 2355000 | 99 | 48 | 1.66E−71 | 1.08E−67 | SPPL2B |
| 61 | 14 | 105412500 | 105420000 | 98 | 48 | 1.66E−71 | 1.08E−67 | AHNAK2 |
| 59 | 3 | 195502500 | 195510000 | 81 | 48 | 1.66E−71 | 1.08E−67 | MUC4 |
| 60 | 5 | 840000 | 847500 | 58 | 48 | 1.66E−71 | 1.08E−67 | ZDHHC11 |
| 62 | 14 | 106342500 | 106350000 | 51 | 48 | 1.66E−71 | 1.08E−67 | IGHD1-26, |
| | | | | | | | | IGHD6-25, |
| | | | | | | | | IGHD5-24 |
| 64 | 2 | 10147500 | 10155000 | 55 | 47 | 1.33E−69 | 8.26E−66 | |
| 65 | 6 | 32542500 | 32550000 | 55 | 47 | 1.33E−69 | 8.26E−66 | HLA-DRB1 |
| 66 | 19 | 54720000 | 54727500 | 48 | 47 | 1.33E−69 | 8.26E−66 | RPS9, |
| | | | | | | | | LILRB3, |
| | | | | | | | | LILRA6 |
| 71 | 16 | 997500 | 1005000 | 104 | 46 | 1.05E−67 | 6.04E−64 | LMF1 |
| 69 | 9 | 117855000 | 117862500 | 57 | 46 | 1.05E−67 | 6.04E−64 | TNC |
| 68 | 6 | 32707500 | 32715000 | 49 | 46 | 1.05E−67 | 6.04E−64 | HLA-DQA2 |
| 67 | 6 | 32467500 | 32475000 | 48 | 46 | 1.05E−67 | 6.04E−64 | |
| 70 | 12 | 104370000 | 104377500 | 47 | 46 | 1.05E−67 | 6.04E−64 | TDG |
| 74 | 7 | 390000 | 397500 | 87 | 45 | 8.04E−66 | 4.16E−62 | |
| 72 | 2 | 1530000 | 1537500 | 76 | 45 | 8.04E−66 | 4.16E−62 | TPO |
| 77 | 15 | 29077500 | 29085000 | 72 | 45 | 8.04E−66 | 4.16E−62 | |
| 78 | 18 | 14430000 | 14437500 | 51 | 45 | 8.04E−66 | 4.16E−62 | |
| 76 | 14 | 106807500 | 106815000 | 50 | 45 | 8.04E−66 | 4.16E−62 | IGHVII-31- |
| | | | | | | | | 1, IGHV3- |
| | | | | | | | | 32 |
| 75 | 7 | 76627500 | 76635000 | 49 | 45 | 8.04E−66 | 4.16E−62 | UPK3B, |
| | | | | | | | | DTX2P1, |
| | | | | | | | | DTX2P1- |
| | | | | | | | | UPK3BP1- |
| | | | | | | | | PMS2P11, |
| | | | | | | | | UPK3BP1 |
| 79 | 19 | 53977500 | 53985000 | 46 | 45 | 8.04E−66 | 4.16E−62 | ZNF813, |
| | | | | | | | | TPM3P6 |
| 73 | 2 | 112305000 | 112312500 | 45 | 45 | 8.04E−66 | 4.16E−62 | |
| 87 | 17 | 165000 | 172500 | 89 | 44 | 6.02E−64 | 2.73E−60 | RPH3AL |
| 86 | 13 | 114000000 | 114007500 | 87 | 44 | 6.02E−64 | 2.73E−60 | GRTP1, |
| | | | | | | | | GRTP1-AS1 |
| 85 | 7 | 142462500 | 142470000 | 50 | 44 | 6.02E−64 | 2.73E−60 | PRSS3P1 |
| 89 | 22 | 22065000 | 22072500 | 50 | 44 | 6.02E−64 | 2.73E−60 | YPEL1 |
| 80 | 1 | 4822500 | 4830000 | 49 | 44 | 6.02E−64 | 2.73E−60 | AJAP1 |
| 88 | 19 | 14505000 | 14512500 | 48 | 44 | 6.02E−64 | 2.73E−60 | CD97 |
| 82 | 3 | 123705000 | 123712500 | 47 | 44 | 6.02E−64 | 2.73E−60 | ROPN1 |
| 81 | 1 | 47610000 | 47617500 | 45 | 44 | 6.02E−64 | 2.73E−60 | CYP4A22- |
| | | | | | | | | AS1, |
| | | | | | | | | CYP4A22 |
| 90 | 22 | 25567500 | 25575000 | 45 | 44 | 6.02E−64 | 2.73E−60 | KIAA1671 |
| 83 | 6 | 3150000 | 3157500 | 44 | 44 | 6.02E−64 | 2.73E−60 | BPHL, |
| | | | | | | | | TUBB2A |
| 84 | 6 | 3172500 | 3180000 | 44 | 44 | 6.02E−64 | 2.73E−60 | TUBB2BP1 |
| 95 | 22 | 20707500 | 20715000 | 84 | 43 | 4.39E−62 | 1.89E−58 | FAM230A, |
| | | | | | | | | USP41 |
| 91 | 1 | 17272500 | 17280000 | 46 | 43 | 4.39E−62 | 1.89E−58 | CROCC |
| 94 | 19 | 53970000 | 53977500 | 46 | 43 | 4.39E−62 | 1.89E−58 | ZNF813 |
| 92 | 2 | 91950000 | 91957500 | 43 | 43 | 4.39E−62 | 1.89E−58 | |
| 93 | 14 | 106200000 | 106207500 | 43 | 43 | 4.39E−62 | 1.89E−58 | IGHG1 |
| 96 | 6 | 29917500 | 29925000 | 150 | 42 | 3.12E−60 | 1.26E−56 | HLA-W |
| 97 | 6 | 169095000 | 169102500 | 47 | 42 | 3.12E−60 | 1.26E−56 | |
| 101 | 19 | 44865000 | 44872500 | 45 | 42 | 3.12E−60 | 1.26E−56 | ZNF112 |
| 99 | 14 | 106935000 | 106942500 | 44 | 42 | 3.12E−60 | 1.26E−56 | LINC00221 |

### SV Distribution in the Most Frequently Affected Genes

The genes that appear most frequently affected by SVs, i.e. the top 10 ranking genes in Table 2, do not comprise the most frequently affected 7.5 kb genomic subregions (Table 3), with the exception of TTC28 (the number 1 ranked bin) and the genomic region containing ZDHHC11 (the number 10 ranked bin). Instead, several genes that are frequently affected by SVs comprise stretches of multiple 7.5 kb genomic subregions that are frequently and significantly affected by SVs. The distribution of SVs in some of the genes that are commonly affected (Table 2) was visualized in IGV Several of these genes, among which FHIT and WWOX, are considered to be located in common fragile sites (Rajaram et al., 2013. PLOSone 8: e66264). The regions affected by SVs are relatively long (>500 kb) and contain many 7.5 kb bins that are significantly affected by SVs, mostly due to deletions. This pattern was observed for MACROD2, FHIT, RBFOX1, PARK2, CCSER1, NAALADL2 and WWOX.

### SV Distribution in the Most Frequently Affected Genomic Subregions

The distribution of SVs in some of the 7.5 kb genomic regions that are commonly affected (Table 2) was visualized in IGV. Of note, some of these genomic regions appear frequently affected by translocations that overlap closely with the position of ‘hot-L1s’, LINE-1 elements that are active in the human (cancer) genome (data not shown). Others contain mostly deletions that may affect long noncoding RNAs.

Recently, an overview of the activity of ‘hot-L1s’ in the cancer genome was provided by a study from the PCAWG consortium (Rodriguez-Martin et al., 2020. Nature Genetics 52:306-319). Strikingly, the top 4-ranked genomic subregions (Table 3) may refer to hot-Lis (see Table 4). Each of these four genomic subregions appears to be predominately affected by translocations. However, instead of actually being the genomic position containing SVs, they rather refer to the position of the genomic source of the ‘hotL1’ that ‘jumps around’ and integrates elsewhere in (multiple distant locations in) the genome where it does introduce new SVs. Sequencing reads that span these newly integrated sites will be interpreted as translocations between the genomic location where the hot-L1 element integrated (the truly newly introduced SVs) and the genomic position where the ‘hot-L1’ source element is located in the reference genome.

### Summarizing Conclusions Example 2

The studies described in example 2 demonstrate:
SVs were mapped at nucleotide resolution using Illumina sequencing-based HMF data.
Aggregation of SVs across 463 CRC samples demonstrated that the genomic position of SVs in cancer is not random.
Using a gene-centric approach, many genes were shown to be frequently affected by SVs, largely overlapping with the same genes that were previously detected using array-based approaches (see Example 1).
Using a binning approach, many small distinct genomic subregions were identified that appear frequently affected by SVs.
A subset of the bins that appear frequently affected by SVs are located near and/or overlapping with ‘hot-L1s’. Hot-L1s represent a distinct mechanism in which SVs are introduced in the cancer genome and may therefore have distinct biomarker potential.

TABLE 4
(A) Comparison of the SV top-ranked bin data from Table 3 with data from the PCAWG consortium paper (Rodriguez-Martin et
al., 2020. Nature Genetics 52: 306-319) reveals significant overlap with the genomic position of ‘hot-L1s’. (B) Supplementary
Table 5 of the Rodriguez-Martin et al., 2020 paper lists 124 hot-L1s; the top 4-ranked genomic regions identified in this
study (see Table 3) overlap very well with entry 56, entry 126, entry 29 and entry 18 of the Supplementary Table 5.
A

| | | 22q12.1 | 14q23.1 | | | Xp22.2-1 | | | | | | Other |
|---|---|---|---|---|---|---|---|---|---|---|---|---|
| | | Rank 1 | Rank 3 | 6p22.1 | 6p24.1 | Rank 2 | 9q32 | 2q24.1 | Xp22.2-2 | 3q21.1 | 7p12.3 | (114) |
| Pan-cancer (2,954) | 3,541 | 22.4 | 9.8 | 7.8 | 5.5 | 5.2 | 3.6 | 3.3 | 3.2 | 2.7 | 2.0 | 34.5 |
| Eso-AdenoCA (99) | 1,582 | 16.4 | 15.3 | 10.7 | 6.4 | 4.9 | 4.6 | 4.2 | 0.9 | 3.9 | 4.0 | 28.7 |
| Head-SCC (57) | 343 | 18.7 | 5.2 | 4.1 | 10.8 | 0.3 | 7.0 | 0.9 | 8.5 | 2.6 | 0.6 | 41.4 |
| Lung-SCC (48) | 297 | 14.5 | 5.7 | 2.0 | 14.5 | 2.0 | 4.7 | 5.4 | 6.1 | 4.0 | 0.0 | 41.1 |
| Panc-AdenoCA (246) | 266 | 35.3 | 5.6 | 13.9 | 1.1 | 10.2 | 4.5 | 0.0 | 0.4 | 1.5 | 0.4 | 27.1 |
| ColoRect-AdenoCA | 229 | 15.7 | 10.5 | 5.7 | 0.0 | 6.1 | 0.9 | 9.2 | 0.4 | 0.9 | 0.9 | 49.8 |
| (62) | | | | | | | | | | | | |
| Prost-AdenoCA (297) | 168 | 34.5 | 0.0 | 1.2 | 1.2 | 9.5 | 0.0 | 0.0 | 18.5 | 0.0 | 0.0 | 35.1 |
| Stomach-AdenoCA | 139 | 25.9 | 8.6 | 0.7 | 3.6 | 12.9 | 1.4 | 3.6 | 0.7 | 3.6 | 0.0 | 38.8 |
| (81) | | | | | | | | | | | | |
| Ovary-AdenoCa (118) | 118 | 16.1 | 11.0 | 9.3 | 0.0 | 9.3 | 0.0 | 0.8 | 2.5 | 0.8 | 2.5 | 47.5 |
| Uterus-AdenoCA (51) | 103 | 40.8 | 0.0 | 8.7 | 3.9 | 2.9 | 0.0 | 0.0 | 5.8 | 0.0 | 0.0 | 37.9 |
| Breast-AdenoCA | 102 | 69.6 | 1.0 | 1.0 | 0.0 | 4.9 | 1.0 | 1.0 | 1.0 | 0.0 | 0.0 | 21.6 |
| (201) | | | | | | | | | | | | |
| Other (1,694) | 125 | 48.8 | 4.8 | 8.8 | 0.0 | 3.2 | 0.8 | 2.4 | 2.4 | 1.6 | 0.0 | 27.2 |

B

Supplementary Table 5 - PCAWG data

| Table 2 | Cytoband_id | coordinates |
|---|---|---|
| Rank 1 | 22q12.1 | 22: 29059272-29065304 |
| Rank 2 | Xp22.2-1 | X: 11725366-11731400 |
| Rank 3 | 14q23.1 | 14: 59220385-59220402 |
| Rank 4 | 12p13.32 | 12: 3608362-3614395 |

### Example 3—the Targeted Detection of SVs

### Materials and Methods

### Targeted Capture of DNA and PacBio Long Read Sequencing

DNA was isolated from 18 tissue samples, comprising 11 colorectal cancers, 4 colorectal adenomas and 3 normal colorectal tissue samples from 13 patients. A targeted capture of DNA was performed using custom designed Roche Nimblegen probes to capture the 19 regions of interest that were recurrently affected by SVs in adenoma-to-carcinoma progression (van den Broek, thesis chapter 5, VU University Medical Center 16 Dec. 2016). In addition the well-known colorectal cancer genes KRAS, BRAF, TP53 and APC were captured for investigation of SNVs, as a positive control (Table 5). Subsequently PacBio CCS sequencing was performed using the PacBio Sequel system (Mount Sinai Hospital, New York City). The alignment and SV analysis using PBSV were done in collaboration with PacBio (Menlo Park, CA), which resulted in the alignment dataset and unfiltered SV dataset.

### Detection and Visualization of Somatic SVs

SV calling was done using the PacBio SV calling and analysis toolkit PBSV (PBSV v2.2.2; available at github.com/PacificBiosciences/pbsv). PBSV is specialized in long read alignment data. An all-inclusive SV detection was performed by making a call whenever two reads deviated from the reference genome. This allowed for the detection of the majority of germline and somatic SVs in the data. Subsequently filtering steps were performed to remove germline SVs, consisting of published germline SVs (Audano et al., 2019. Cell 176:663-675) combined with the SV calls from normal colorectal tissue samples. In addition, coverage of tumor and normal samples must have sufficient read depth to make reliable SV calls. Examples of the structural variants detected with PacBio long read sequencing were visualized using Ribbon (Nattestad et al., 2020. Bioinformatics btaa680).

### Results

### Main Objectives

We previously demonstrated detection of SCNA-associated SVs based on SCNA calls derived from array-based (or shallow whole genome sequencing-based) approaches (example 1) and detection of SVs at nucleotide resolution by HMF using Illumina whole genome sequencing (example 2). Knowing what regions of the genome are frequently affected by SVs in cancer, we now wanted to demonstrate that targeted sequencing of these regions in tumor samples can be used to detect somatic SVs. In particular, it is important to demonstrate that capture-based approaches that are designed to capture the genomic regions that comprise SVs is also capable to retrieve for sequencing the genomic segments to which these SVs connected. We aimed to investigate:

### 1. Targeted Detection of SVs Using PacBio Long Read Sequencing

### Targeted Detection and Visualization of SVs in Adenomas and CRCs

Using targeted PacBio CCS long read sequencing we succeeded to detect SVs in CRC and adenoma samples, in 6 different genes: MACROD2, WWOX, FHIT, PRKN, NOTCH2 and RAD51B (Table 6). NOTCH2 was affected by one SV in 4 out of 11 CRC samples. MACROD2 was affected by SVs in 8 out of 11 CRC samples and in 1 adenoma. In 6 of these cases more than one SV was detected in MACROD2, up to 34 SVs in one sample. Also for WWOX, FHIT, PARKN (also known as PARK2) and RAD51B there were tissue samples with multiple SVs in these genes. It remains to be determined whether the number of SVs in these preselected genes is related to the TBL, in which case the TBL may be estimated based on the detailed SV analysis of a few preselected genes.

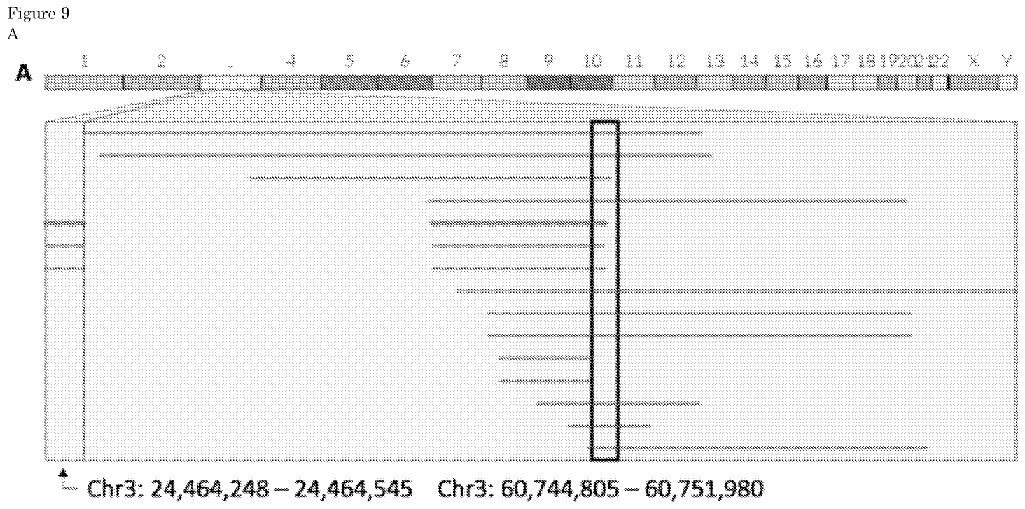

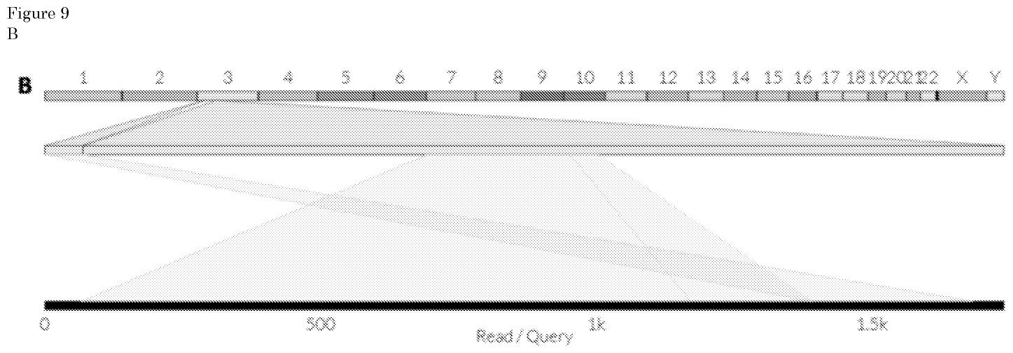

One advantage of long read sequencing compared to Illumina sequencing is the feasibility to visualize complex rearrangements that span hundreds of basepairs at nucleotide resolution. SVs that are detected by PacBio long read sequencing can be visualized by Ribbon. In FIG. 9 an example is shown of detection of an SV in FHIT on chromosome 3. Ribbon shows the exact position where the SV call was made as well as where the reads that support the SV ‘jump’ to in the genome. In this particular example the FHIT gene is truncated to only its first 5 exons, which likely affects its function.

### Summarizing Conclusions Example 3

The studies described in Example 3 demonstrate:
Targeting preselected genomic regions for detection of SVs is possible.
Detection of SVs by long read sequencing allows to characterize the SV event at nucleotide resolution, thereby providing one or more personalized markers that are specific for said tumor of the patient.
Several of the genes we selected revealed multiple SVs in a single tumor sample, probably because they represent fragile sites.

TABLE 5
Overview of the genes/genomic regions that were captured
for targeted PacBio long read sequencing using custom
designed Roche Nimblegen probes. The 19 regions of interest
were selected because they are rarely being affected
by SVs in colorectal adenomas while frequently being
affected by SVs in CRC (van den Broek, thesis chapter
5, VU University Medical Center 16 Dec. 2016). The 4
genes APC, TP53, KRAS and BRAF are commonly mutated
by SNVs in CRC and were taken along as ‘positive controls’
for detecting somatic alterations.

| Region | Genetic | Region | |
|---|---|---|---|
| number | location | length (kb) | Genes present |
| 1 | 20p12.1 | 2085.2 | MACROD2 |
| 2 | 20q13 | 88.7 | PCMTD2, LINC00266-1 |
| 3 | 6q26 | 1995.7 | PRKN, PACRG |
| 4 | 3p14.2 | 1539.8 | FHIT |
| 5 | 1p12 | 236.2 | NOTCH2 |
| 6 | 11q13 | 214.7 | FRMD8, MALAT |
| 7 | 16q23.1 | 1173 | WWOX |
| 8 | 14q24.1 | 927.9 | RAD51B |
| 9 | 2p21 | 205.3 | EPCAM, MSH2 |
| 10 | 13q21 | 350.4 | DIS3, PIBF1, KLF5 |
| 11 | 15q25.3 | 435.9 | AKAP13 |
| 12 | 18q11.1 | 184.6 | ROCK1 |
| 13 | 14q32.33 | 80.1 | MTA1 |
| 14 | 6p25.3 | 692.9 | GMDS |
| 15 | 6p22.33 | 659.5 | PTPRK |
| 16 | 6p22 | 150.9 | TRIM38, HIST1H1A |
| 17 | 17q21.2 | 146.8 | STAT5B |
| 18 | 12p13 | 119.7 | CLEC4A, ZNF705A |
| 19 | 8q24.3 | 157 | ZC3H3 |
| A | 5q22.2 | 171.2 | APC |
| B | 17p13.1 | 39.2 | TP53 |
| C | 12p12.1 | 64.9 | KRAS |
| D | 7q34 | 214.3 | BRAF |

TABLE 6
Overview of SVs that were detected in CRC and adenoma samples using
targeted PacBio CCS long read sequencing. After filtering germline
variation and low-confidence somatic calls, somatic SVs were called
in six different genes: MACROD2, WWOX, FHIT, PRKN, NOTCH2 and RAD51B.

| Patient ID | Sample type | MACROD2 | WWOX | FHIT | PRKN | NOTCH2 | RAD51B |
|---|---|---|---|---|---|---|---|
| 005 | Carcinoma | 8 | 8 | 7 | 10 | 1 | 7 |
| 031 | Carcinoma | 1 | 0 | 1 | 3 | 0 | 0 |
| 034 | Carcinoma | 0 | 1 | 1 | 11 | 0 | 2 |
| 038 | Carcinoma | 0 | 0 | 0 | 0 | 0 | 0 |
| 050 | Carcinoma | 4 | 0 | 2 | 4 | 0 | 0 |
| 057 | Carcinoma | 34 | 13 | 10 | 32 | 0 | 5 |
| 061 | Carcinoma | 1 | 0 | 0 | 1 | 1 | 0 |
| 064 | Carcinoma | 3 | 1 | 3 | 3 | 0 | 0 |
| 066 | Carcinoma | 16 | 3 | 8 | 37 | 1 | 3 |
| 070 | Carcinoma | 2 | 0 | 0 | 2 | 0 | 0 |
| 072 | Carcinoma | 0 | 0 | 5 | 0 | 1 | 0 |
| 004 | Adenoma | 1 | 0 | 1 | 0 | 0 | 0 |
| 044 | Adenoma | 0 | 0 | 1 | 6 | 0 | 0 |

### Example 4-Impact of MACROD2 on the Tumor Break Load and Prognosis

### Materials and Methods

The same methodology and methods have been used as described in Example 1 herein above, including
Public data from The Cancer Genome Atlas;
MSI status analysis;
Somatic Copy Number Alterations (SCNA) status analysis;
Detection of SCNA-associated SVs;
Determination of Tumor Break Load (TBL); and
Building a TBL classifier.
   In addition public data from the Hartwig Medical Foundation was used.

A data set of 616 stage IV CRC biopsies originating from 607 individual patients was provided by the Hartwig Medical Foundation (HMF) (Priestley et al., 2019. Nature 575:210-216). The biopsies were taken from the metastatic lesions. The data set contained clinical data, raw Whole Genome Sequencing (Illumina HiSeq X-Ten) reads of tumor and patient-matched controls, and structural variant calls. The HMF used their GRIDSS-PURPLE-LINX pipeline (Cameron et al., 2019. bioRxiv 781013) to call structural variants. Matching normal blood samples were used to correct for germline variants. Ensembl GRCh37 (Cunningham et al., 2019. Nucleic Acids Res 47: D745-D751) was used as the reference genome.

### Survival Analysis

Patients were classified into two DNA-based Tumor Break Load (TBL) groups: TBL-High and TBL-Low. The optimal threshold to divide the patients into ‘TBL-High’ and ‘TBL-Low’ was determined using the Maximally Selected Rank Statistic (maxstat) (Hothorn, 2017. maxstat: R package version 0.7-25; see available at//CRAN.R-project.org/package=maxstat) that determines the optimal cut-off resulting in maximal differentiation between the two groups. Kaplan-Meier survival analysis and Cox proportional-hazard analysis were conducted on the classified patient groups (TBL-High and TBL-Low) to visualize and calculate the significance of differences between the classified TBL-High and TBL-Low patients. The Kaplan-Meier survival plots were visualized using the R package survminer (Kassambara et al., 2020. R package version 0.4.8; available at CRAN.R-project.org/package=survminer). Log-rank test P-values and Hazard ratios were calculated using the R package survival (version 3.2.7, Therneau and Grambsch 2000. Modeling Survival Data: Extending the Cox Model. Springer Verlag, New York. ISBN 0-387-98784-3). Kaplan-Meier curves were generated using public data from The Cancer Genome Atlas and public data from 66 stage II and III colorectal cancer patients reported by Orsetti et al., 2014 who did not receive adjuvant treatment (Orsetti, et al., 2014. BMC Cancer 14, 121).

### Results

### Main Objectives

We demonstrated in Example 1 that classification of patients into low- and high-RNA-based ‘predicted TBL’ show that TBL has prognostic value for disease recurrence (FIGS. 4A and 4B) and that SVs in the gene MACROD2 are associated with an increased TBL (FIG. 6).

We now investigated (1) a prognostic value of DNA-based TBL; and (2) the impact of SVs within MACROD2 on TBL

### (1) DNA-Based TBL has Prognostic Value.

Patients with localized or loco-regional (stage I, II or III) CRC receive surgery to remove the primary tumor. A substantial proportion of these patients (more than 50%) is cured by surgery alone, while another substantial proportion of these patients has minimal residual disease that can lead to disease recurrence (up to 50%, pending stage). Stratifying for patients at high risk of disease recurrence will facilitate selection of patients who need adjuvant therapy. We demonstrated that RNA-based predicted TBL has prognostic value (see Example 1). We now investigated whether DNA-based TBL can be used directly as a prognostic biomarker while focusing on the patient population for whom this question is clinically most relevant, i.e. patients with stage II or III MSS CRC.

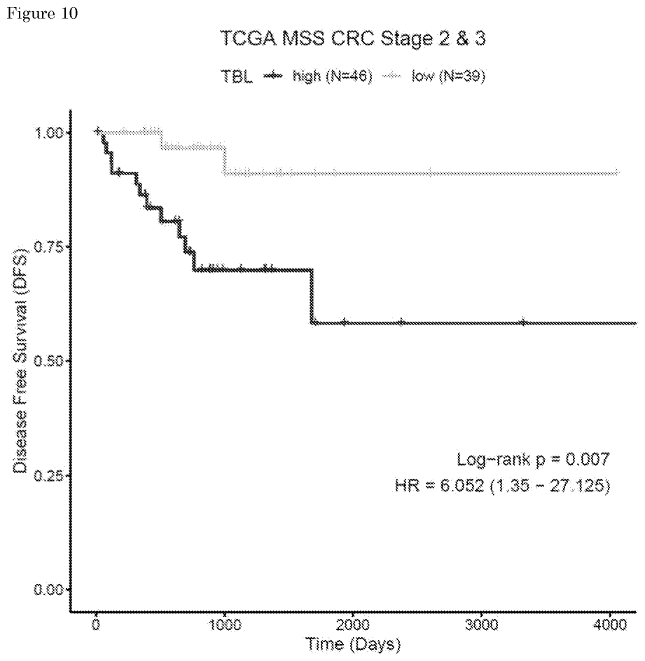

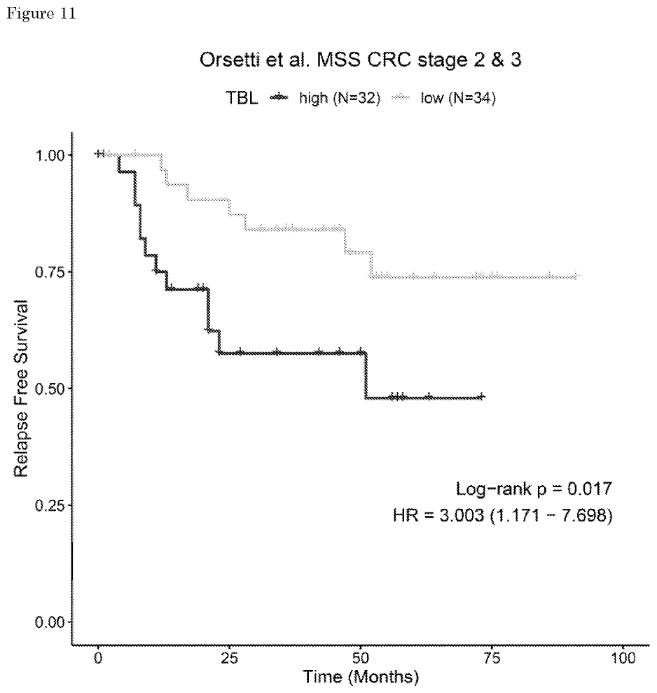

First, we examined this subset of patients using data from The Cancer Genome Atlas. By selecting a TBL cut-off point with maximal power to visualize differences in disease free survival (see Methods section), stage II-III TCGA COADREAD MSS patients that were TBL-high were shown to have a significantly worse prognosis compared to TBL-low patients (P<0.01). Even more importantly, the clinical effect size indicated by the Hazard Rate was very high (HR>6). The Kaplan-Meier curve is presented in FIG. 10. Next, to validate this finding, we made use of another public dataset to examine the prognostic value of TBL in stage II and III MSS CRC patients (Orsetti et al., 2014. BMC Cancer 14, 121). Again, TBL-High was significantly associated with worse relapse free survival, with a large clinical effect size (p<0.02; HR>3; FIG. 11).

(2) SVs that Affect MACROD2 Exon Sequences are Associated with High TBL

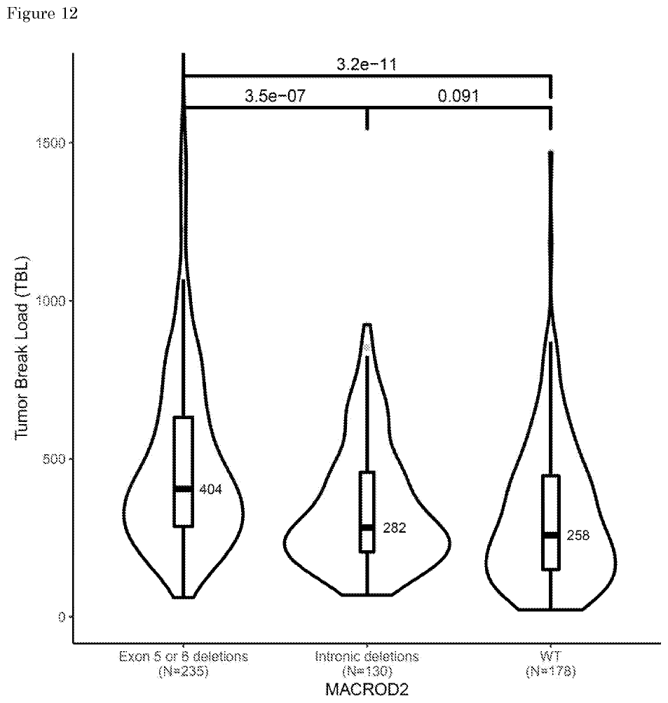

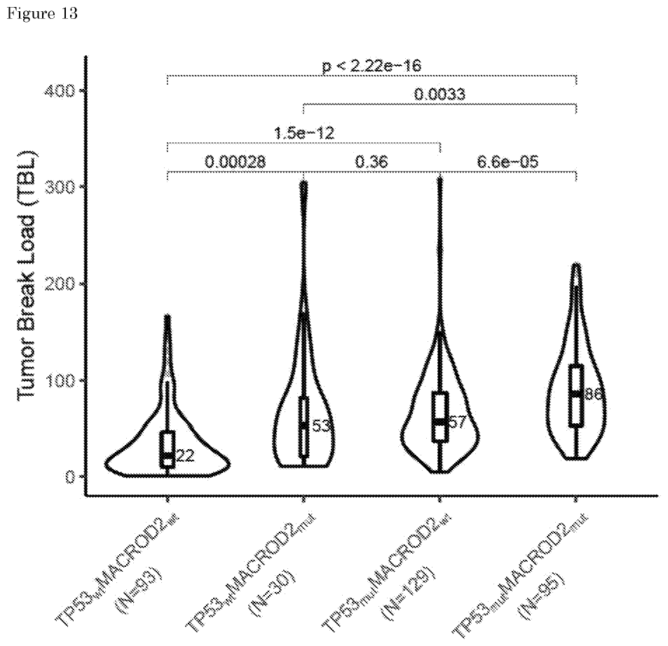

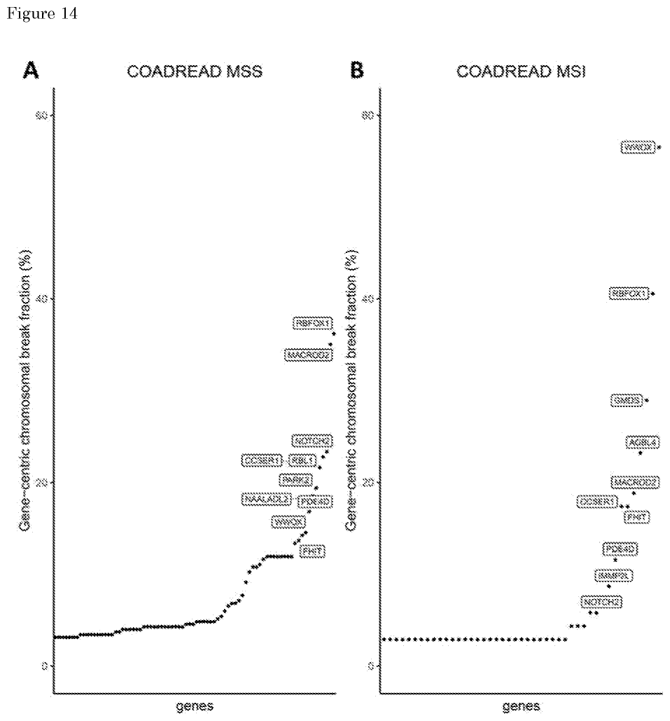

MACROD2 plays a role in Double Strand Break repair. We demonstrated in example 1 that SCNA-associated SVs in MACROD2 are associated with a higher TBL (FIG. 6). However, MACROD2 is a very large gene (approximately 2 megabases), in which a substantial proportion of SVs may be positioned in intronic regions. We now investigated the position of SVs, in particular focal deletions, within the MACROD2 gene using HMF data, in which SVs were detected at nucleotide resolution. In 24% of these samples SVs were observed in an intronic region in MACROD2, mostly between exon 5 and exon 6. In 43% of samples SVs in MACROD2 did affect exonic sequences, in particular exon 5 and/or exon 6. Strikingly, the TBL of samples with intronic MACROD2 alterations was similar to that of MACROD2-wildtype samples, while samples with exonic alterations in MACROD2 had a significantly higher TBL (FIG. 12).

Somatic alterations in TP53 are the most prevalent in cancer across all cancer types. TP53 mutations are known to facilitate chromosomal instability. We examined whether the effect of MACROD2 on TBL was present irrespective of TP53 alterations, using stage I-IV MSS TCGA COADREAD patients. Alterations in MACROD2 or TP53 were associated with a significantly higher TBL than without alterations in either of these genes. The effect on TBL was highest when both MACROD2 and TP53 were mutated (FIG. 12).

### Summarizing Conclusions Example 4

The studies described in Example 4 demonstrate that DNA-based TBL has clinically relevant prognostic value patients with localized (colorectal) cancer. This means that DNA-based TBL can be used to stratify patients after surgery for additional adjuvant (chemo) therapy.

These studies further demonstrate that SVs that affect exon sequences in MACROD2 are associated with a high TBL while SVs that only affect MACROD2 intronic sequences do not. This means that SVs within MACROD2 must be detected at a resolution that is sufficient to determine whether MACROD2 exons are affected or not in order to determine whether MACROD2 function in Double Strand Break repair is affected or not. This further means that the position of SVs within MACROD2 will guide the choice of ((neo) adjuvant) therapy, e.g. fluoro-uracil-based chemotherapy.

These studies further demonstrate that SVs in MACROD2 are not redundant with TP53 mutations.

### Example 5—CRC Genomic Instability-Specific SV-Mutated Genes

### Materials and Methods

The same methodology and methods have been used as described in Example 1 herein above. To identify genes that are differentially affected by SVs in distinct tumor (sub) types, a chi-square (X2) was used. To correct for multiple hypothesis testing, a Benjamini-Hochberg correction (Benjamini and Hochberg, 1995. J R Stat Soc B 57:289-300) was applied.

### Results

### Main Objectives

We previously demonstrated in example 1 that the distribution of the frequencies in which these genes are affected by SVs differs among cancer types (CRC, BRCA and LUAD; FIG. 5), implying that the biological impact of these SV alterations are, at least to some extent, cancer type specific. We now investigated the frequencies of SVs in CRC with distinct genomic instability, MSS versus MSI.

### SVs in WWOX and GMDS are Overrepresented in MSI Tumors Compared to MSS Tumors

MSI and chromosomal instability are the two main types of genomic instability in CRC. MSI tumors have a relatively high tumor mutational burden (TMB) and a relatively low TBL compared to MSS tumors. Therefore, the frequency in which genes are affected by SVs is expected to be lower in MSI tumors compared to MSS tumors, unless there is a selective advantage for such SVs in certain genes. We ranked genes by the frequencies in which they are affected by (SCNA-associated) SVs, using TCGA COADREAD MSS tumors (excluding MSI as well as POLD/E mutants; FIG. 12 [panel A]) and TCGA COADREAD MSI tumors (FIG. 12 [panel B]). For several genes, such as MACROD2, the frequency in which it is affected by SVs in MSS tumors is higher than in MSI tumors. For others, such as RBFOX1, the frequency in which it is affected by SVs in MSS and MSI tumors is similar. Surprisingly, there are also genes like WWOX and GMDS that are more frequently affected by SVs in MSI tumors compared to MSS (FIG. 12).

Statistical evaluation of the prevalence of genes affected by SVs in MSS versus MSI tumors confirmed overrepresentation of SVs in MSI compared to MSS COADREAD tumors for WWOX and GMDS (Table I), implying that alterations in these genes are biologically relevant in MSI tumors, e.g. by contributing to evading the immune response to these TMB-high lesions. Likewise, SVs in RBL1, PARK2 and NOTCH2 are the most overrepresented in MSS tumors compared to MSI tumors (Table 7), implying that these genes contribute functionally to chromosomal instability. Genes that are differentially affected by SVs in MSS versus MSI CRC samples are listed in Table 7.

### Summarizing Conclusions Example 5

The studies described this example demonstrate that the prevalence of SVs in genes in MSS CRC differs from that in MSI CRC, and that SV-alterations in WWOX and GMDS are overrepresented in MSI CRC despite the general low TBL of MSI tumors.

This means that SVs in WWOX and GMDS may provide a selective advantage to MSI tumors. Considering these tumors are well recognized by the immune system due to their high TMB, it is likely that SVs in these genes help to evade the immune response. Consequently, due to the functional effects of SVs in WWOX and GMDS these alterations can be indicative for selecting patients for immunotherapy. This stratification for immunotherapy is not limited to MSI tumors, but also applicable to MSS tumors.

### Example 6—Locations of Hot-L1s

### Materials and Methods

Public Data from the Hartwig Medical Foundation

A data set of 616 stage IV CRC biopsies originating from 607 individual patients was provided by the Hartwig Medical Foundation (HMF) (Priestley et al., 2019. Nature 575:210-216). The biopsies were taken from the metastatic lesions. The data set contained clinical data, raw Whole Genome Sequencing (Illumina HiSeq X-Ten) reads of tumor and patient-matched normals, and structural variant calls. The HMF used their GRIDSS-PURPLE-LINX pipeline (Cameron et al., 2019. bioRxiv 781013) to call structural variants. Matching normal blood samples were used to correct for germline variants. Ensembl GRCh37 (Cunningham et al., 2019. Nucleic Acids Res 47: D745-D751) was used as the reference genome.

### Identification of Hot-L1 Genomic Regions and Translocations

The coverage of translocating events called by the GRIDSS-PURPLE-LINX pipeline provided by HMF was used to identify genomic regions more frequently affected by translocations than the average genomic position in the genome. Genomic regions with a peak minimal translocation coverage of 35 were considered hot-L1 regions. The identified genomic regions were compared to known L1 locations reported by Rodriguez-Martin, B. et al. (Rodriguez-Martin et al., 2020. Nature Gen 52:306-319). The genomic coordinates of the source (hot-L1) and destination (integration site) were annotated to genes using the genomic coordinates from Ensembl GRCh37. The gene annotation of the destinations was used to calculate the fraction of translocations that is located within genes.

### Results

### Main Objectives

We previously demonstrated in Example 2 that four of the most affected 7.5 kb-binned regions overlap with active hot-L1 regions (Table 4B).

We now investigated the activity of Hot-L1 activity in CRC.

Frequent Activation and Abundant Translocation of Hot-L1s into Genes

Recently, Rodriguez-Martin et al. (2020) reported the locations of LINE1 elements that frequently become activated in cancer. We explored the activity of Hot-L1s in CRC using HMF data and list the genomic positions of the 19 most active Hot-L1 elements in CRC; the proportion of CRCs in which each of these Hot-Lis is activated; the number of translocations observed for each Hot-L1 element; and the percentage of cases in which they integrate in gene sequences (see Table 8). Of the 19 regions identified, 15 regions overlap with the previously reported active hot-L1 regions. We also show that a high proportion of translocation events are integrated within genes. Additionally, we show that these identified active hot-Lis show different global and sample specific activity levels (Table 8).

### Summarizing Conclusions Example 6

The studies described in this example demonstrate that the top 19 most active hot-Lis are active in CRC, indicating that a large proportion of CRCs has active hot-L1 elements. This means that a small ‘footprint’ of the genome can be targeted for detecting many tumor-specific SV biomarkers. The studies described in this example further demonstrate the frequent integration of Hot-L1s within gene sequences. This means that Hot-L1 translocations are likely affecting gene function in carcinogenesis, which may impact tumor biology and patient treatment options.

TABLE 7
Top 20 genes that are most differentially affected by SVs (chromosomal
breaks) in MSS versus MSI TCGA COADREAD patients.

| | Fraction | Fraction MSI | | |
|---|---|---|---|---|
| | MSS CRCs | CRCs | | |
| | affected by | affected by | | |
| Gene | SVs (%) | SVs (%) | p-value | FDR |
| WWOX | 14.5% | 56.5% | 2.73E−14 | 3.73E−11 |
| GMDS | 4.8% | 29.0% | 4.49E−10 | 3.07E−07 |
| RBL1 | 22.8% | 0.0% | 2.24E−05 | 0.010189 |
| PARK2 | 19.4% | 4.3% | 0.004123 | 1 |
| NOTCH2 | 23.4% | 7.2% | 0.004274 | 1 |
| FRG1B | 11.7% | 0.0% | 0.005662 | 1 |
| PRKG1 | 13.4% | 1.4% | 0.008215 | 1 |
| HCN1 | 10.8% | 0.0% | 0.008381 | 1 |
| MACROD2 | 35.0% | 18.8% | 0.012824 | 1 |
| NAALADL2 | 18.5% | 5.8% | 0.015123 | 1 |
| PACRG | 11.1% | 1.4% | 0.022899 | 1 |
| PARP8 | 10.8% | 1.4% | 0.025978 | 1 |
| MROH8 | 10.3% | 1.4% | 0.033396 | 1 |
| AC241377.2 | 12.0% | 2.9% | 0.04202 | 1 |
| AC241585.2 | 12.0% | 2.9% | 0.04202 | 1 |
| AC243756.1 | 12.0% | 2.9% | 0.04202 | 1 |
| CH17.270A2.2 | 12.0% | 2.9% | 0.04202 | 1 |
| HFE2 | 12.0% | 2.9% | 0.04202 | 1 |
| HIST2H3PS2 | 12.0% | 2.9% | 0.04202 | 1 |
| NOTCH2NL | 12.0% | 2.9% | 0.04202 | 1 |

TABLE 8
Overview table of 19 active Hot-L1 regions and their characteristics in metastatic colorectal cancer
lesions (HMF data). The Table lists the Hot-L1 locations in HMF data (Genome build: GRCh37) and
the reported location from literature (Rodriguez-Martin et al., 2020. Nature Genetics 52: 306-319);
annotation if the source L1 is positioned within a gene; the total number of translocations that
originate from the L1; the percentage of translocations that end up in gene sequences; and the
percentage of samples affected by L1 translocation (total number of samples N = 616).

| | L1 position | Reported | | | Fraction of | Percentage |
|---|---|---|---|---|---|---|
| | within | L1 | Gene | Number of | translocations in | affected samples |
| Chromosome | chromosome | location* | annotation | Translocations | genes (%) | (N = 616) |
| 1 | 119300000- | 119394975- | outside | 1215 | 39.26 | 15.7 |
| | 119499999 | 119401004 | | | | |
| 1 | 199400000- | 121274033- | outside | 711 | 34.04 | 31.7 |
| | 199499999 | 121280060 | | | | |
| 1 | 87100000- | 87144764- | CLCA4-AS1 | 262 | 42.37 | 8.8 |
| | 87199999 | 87150795 | | | | |
| 2 | 156500000- | 156527822- | outside | 480 | 23.12 | 5.2 |
| | 156599999 | 156527848 | | | | |
| 2 | 88200000- | not | outside | 137 | 24.09 | 12.3 |
| | 88299999 | reported | | | | |
| 3 | 123590000- | 123590713- | MYLK | 2598 | 31.72 | 15.3 |
| | 123599999 | 123590726 | | | | |
| 4 | 19070000- | not | outside | 2035 | 26.63 | 12.0 |
| | 19079999 | reported | | | | |
| 6 | 29920000- | 29920213- | outside | 2308 | 32.84 | 3.1 |
| | 29920999 | 29920223 | | | | |
| 6 | 13190000- | 13191016- | PHACTR1 | 2294 | 23.93 | 11.2 |
| | 13199999 | 13191033 | | | | |
| 7 | 47000000- | 47864826- | PKD1L1 | 1932 | 30.64 | 4.4 |
| | 47999999 | 47864838 | | | | |
| 7 | 30470000- | 30478858- | NOD1 | 375 | 31.73 | 7.0 |
| | 30489999 | 30484915 | | | | |
| 9 | 115500000- | 115560408- | SNX30 | 1449 | 31.54 | 11.0 |
| | 115599999 | 115566440 | | | | |
| 10 | 111570000- | not | outside | 99 | 35.35 | 6.8 |
| | 111589999 | reported | | | | |
| 12 | 3600000- | 3608362- | PRMT8 | 1573 | 22.7 | 26.6 |
| | 3619999 | 3614395 | | | | |
| 14 | 59220000- | 59220385- | LINC01500 | 18152 | 35.09 | 22.9 |
| | 59229999 | 59220402 | | | | |
| 15 | 55200000- | 56251135- | NEDD4 | 532 | 32.89 | 24.8 |
| | 56259999 | 56251152 | | | | |
| 22 | 29050000- | 29059272- | TTC28 | 34971 | 35.76 | 68.3 |
| | 29069999 | 29065304 | | | | |
| X | 11720000- | 11725366- | AC004554.2 | 8518 | 20.69 | 31.0 |
| | 11739999 | 11731400 | | | | |
| Y | 9400000- | not | outside | 443 | 25.51 | 8.0 |
| | 9499999 | reported | | | | |

*Rodriguez-Martin et al., 2020. Nature Genetics 52: 306-319

1. A method for tumor marker analysis comprising
providing a genomic DNA sample from tumor cells of a patient;
preselecting a chromosomal region on the genomic DNA comprising at least part of a potential structural variant (SV);
sequencing the genomic region surrounding the potential SV, to provide one or more novel tumor markers that are specific for said tumor of the patient.

2. The method according to claim 1, wherein the genomic DNA sample is obtained from fixed tissue, such as formalin-fixed paraffin-embedded (FFPE) tissue.

3. The method according to claim 1, wherein the step of preselecting is performed by capturing and isolating the chromosomal region comprising at least part of a potential structural variant (SV).

4. The method according to claim 1, wherein the step of preselecting is performed by Targeted Locus Amplification (TLA).

5. The method according to claim 1, wherein the genomic region surrounding the potential SV is sequenced by third generation sequencing.

6. The method according to claim 1, wherein the potential SV is caused by activation of a retrotransposon, preferably a LINE1 element, preferably a hot-L1 element.

7. The method according to claim 1, wherein the potential SV comprises a region on chromosome 22, from nucleotide 29062500 to nucleotide 29070000; a region on chromosome 23, from nucleotide 11730000 to nucleotide 11737500; a region on chromosome 14, from nucleotide 59220000 to nucleotide 59227500; a region on chromosome 12, from nucleotide 3607500 to nucleotide 3615000; a region on chromosome 7, from nucleotide 57442500 to nucleotide 57450000; a region on chromosome 8, from nucleotide 143955000 to nucleotide 143962500; a region on chromosome 9, from nucleotide 139995000 to nucleotide 140002500; a region on chromosome 12, from nucleotide 132060000 to nucleotide 132067500; a region on chromosome 6, from nucleotide 170482500 to nucleotide 170490000; a region on chromosome 5, from nucleotide 742500 to nucleotide 750000, or a combination thereof.

8. The method according to claim 1, wherein the potential SV is a recombination hotspot.

9. The method according to claim 1, wherein the potential SV is a recombination hotspot within MACROD2, FHIT, RBFOX1, PARK2, TTC28, NOTCH2, PIBF1, CCSER1, PTPRN2, NAALADL2, WWOX, or PRKG1.

10. A method of typing a sample from a cancer patient, the method comprising:
providing a sample comprising nucleic acids from said cancer cells;
determining a number of structural variants (SV) in said sample;
comparing said number of SV to a number of SV in a reference; and
typing said sample based on the comparison of the number of SV.

11. The method of claim 10, further comprising determining presence or absence of an SV that affects exon sequences of MACROD2, and/or presence or absence of mutations in TP53.

12. A method for monitoring tumor progression in a patient, comprising
identifying one or more novel tumor markers that are specific for said tumor of the patient as a structural variant (SV) in the patient by performing the method of claim 1, whereby the SV is characterized by a region of at least 20 nucleotides at either site of the SV's associated chromosomal breakpoint,
providing a first biopsy from the patient,
analyzing the first biopsy for presence and/or abundance of the SV,
providing a second biopsy from the patient, whereby the provision of the second biopsy is separated in time or location from the provision of the first biopsy,
analyzing the second biopsy for presence and/or abundance of the SV, and
recording and comparing presence and/or abundance of the SV in the two biopsies.

13. The method of claim 12, wherein the biopsy is a liquid biopsy, preferably a blood sample.

14. The method of claim 12, wherein the patient is treated by therapy between the first and second biopsy.

15. The method of claim 14, whereby the therapy is selected from surgery, chemotherapy, radiation therapy, targeted therapy, immunotherapy, stem cell or bone marrow transplant, hormone therapy, or a combination thereof.

16. A method of treating of a cancer patient with a structural variant (SV) in at least one of WWOX, GMDS, FHIT and PIBF1, said method comprising an immunotherapy by administering an immune checkpoint inhibitor including a PD1/PDL1 inhibitor and/or a CTLA-4 inhibitor.

17. A method of treating a cancer patient with a SV in MACROD2, said method comprising administering to the patient a therapeutically effective amount of Capecitabine and oxaliplatin, combined with a vascular endothelial growth factor inhibitor including bevacizumab, ziv-aflibercept, or ramucirumab, an epidermal growth factor receptor inhibitor including cetuximab or panitumumab, irinotecan, trifluridine and tipiracil, or a combination thereof wherein the SV affects exon sequences of MACROD2.
